SEQUENCE LISTING (10 sequences)

SEQ ID NO: 1 - protein, 18 aa, Synthetic construct
DSSDSSDSSDSSDSSDSS

SEQ ID NO: 2 - protein, 333 aa, synthetic construct
MAVRRDSVWKYCWGVLMVLCRTAISKSIVLEPIYWNSSNSKFLPGQGLVLYPQIGDKLDIICPKVDSKTVGQYEYYKVYMVDKDQADRCTIKKENTPLLNCAKPDQDIKFTIKFQEFSPNLWGLEFQKNKDYYIISTSNGSLEGLDNQEGGVCQTRAMKILMKVGQDASSAGSTRNKDPTRRPELEAGTNGRSSTTSPFVKPNPGSSTDGNSAGHSGNNILGSEVALFAGIASGCIIFIVIIITLVVLLLKYRRRHRKHSPQHTTTLSLSTLATPKRSGNNNGSEPSDIIIPLRTADSVFCPHYEKVSGDYGHPVYIVQEMPPQSPANIYYKV

SEQ ID NO: 3 - protein, 302 aa, synthetic construct
MAVRRDSVWKYCWGVLMVLCRTAISKSIVLEPIYWNSSNSKFLPGQGLVLYPQIGDKLDIICPKVDSKTVGQYEYYKVYMVDKDQADRCTIKKENTPLLNCAKPDQDIKFTIKFQEFSPNLWGLEFQKNKDYYIIYASSAGSTRNKDPTRRPELEAGTNGRSSTTSPFVKPNPGSSTDGNSAGHSGNNILGSEVALFAGIASGCIIFIVIIITLVVLLLKYRRRHRKHSPQHTTTLSLSTLATPKRSGNNNGSEPSDIIIPLRTADSVFCPHYEKVSGDYGHPVYIVQEMPPQSPANIYYKV

SEQ ID NO: 4 - protein, 295 aa, synthetic construct
MAVRRDSVWKYCWGVLMVLCRTAISKSIVLEPIYWNSSNSKFLPGQGLVLYPQIGDKLDIICPKVDSKTVGQYEYYKVYMVDKDQADRCTIKKENTPLLNCAKPDQDIKFTIKFQEFSPNLWGLEFQKNKDYYIISTSNGSLEGLDNQEGGVCQTRAMKILMKVGQGSSTDGNSAGHSGNNILGSEVALFAGIASGCIIFIVIIITLVVLLLKYRRRHRKHSPQHTTTLSLSTLATPKRSGNNNGSEPSDIIIPLRTADSVFCPHYEKVSGDYGHPVYIVQEMPPQSPANIYYKV

SEQ ID NO: 5 - protein, 333 aa, synthetic construct; identical to SEQ ID NO: 2

SEQ ID NO: 6 - protein, 8 aa, synthetic construct
DDDDDDDD

SEQ ID NO: 7 - protein, 14 aa, synthetic construct
DDDDDDDDDDDDDD

SEQ ID NO: 8 - protein, 18 aa, synthetic construct
DSSDSSDSSDSSDSSDSS

SEQ ID NO: 9 - protein, 8 aa, synthetic construct
KNFQSRSH

SEQ ID NO: 10 - protein, 5 aa, synthetic construct
SDSSD

(19) United States
(12) Patent Application Publication
(10) Pub. No.: US 2026/0263364 A1
(43) Pub. Date: Sep. 10, 2026

(54) OSTEOCLAST-DERIVED MICROVESICLES AND METHODS OF PROMOTING BONE REGENERATION

(71) Applicants: The United States Government as represented by the Department of Veterans Affairs, Washington, DC (US); Loma Linda University Health, Loma Linda, CA (US)

(72) Inventors: Kin-Hing William Lau, Loma Linda, CA (US); Han-Chin Matilda Sheng, Loma Linda, CA (US)

(21) Appl. No.: 19/491,432

(22) PCT Filed: Jun. 7, 2024
(86) PCT No.: PCT/US2024/033042

§ 371 Date: Dec. 9, 2025

Related U.S. Application Data
(60) Provisional application No. 63/507,167, filed on Jun. 9, 2023.

(51) Int. Cl. A61K 9/1271; A61K 38/00; C07K 14/47

(52) U.S. Cl. CPC A61K 9/1271; C07K 14/4702; A61K 38/00

(57) ABSTRACT

Disclosed herein are compositions comprising one or more liposomes, and a targeting peptide, wherein the one or more liposomes comprise a transmembrane protein, and wherein the targeting peptide is outside of the liposome. Also disclosed herein are compositions comprising, an osteoclast-derived microvesicle, and a targeting peptide, wherein the osteoclast-derived microvesicle comprises a transmembrane protein, and wherein the targeting peptide is outside the osteoclast-derived microvesicle. The compositions can be used in methods of treating osteoarthritis, post-traumatic osteoarthritis, or skeletal fractures in subjects.

### CROSS REFERENCE TO RELATED APPLICATIONS

This application claims the benefit of the filing date of U.S. Provisional Application No. 63/507,167, filed on Jun. 9, 2023. The content of this earlier filed application is hereby incorporated by reference in its entirety.

### STATEMENT REGARDING FEDERALLY FUNDED RESEARCH

This invention was made with government support under grant numbers IO1BX002964 and IK6BX003782 awarded by the U.S. Department of Veterans Affairs and HT9425-23-1-0061 awarded by the U.S. Department of Defense. The government has certain rights in the invention.

### REFERENCE TO A SEQUENCE LISTING

The present application contains a Sequence Listing that is submitted concurrent with the filing of this application in XML format, containing the file name “37759_0524P1_SL.xml,” created on Jun. 6, 2024, and having a size of 12,288 bytes. The Sequence Listing is hereby incorporated by reference into the present application in its entirety.

### BACKGROUND

Injuries to the joint, chronic or excessive impact loading on the joint, joint dislocations, intra-articular fractures, or genetic predisposition can often lead to lesions in the articular cartilage and cause synovial inflammation (Martin, J. A., and Buckwalter, J. A. (2006) Biorheology 43, 517-521). Repetitive use of the injured joint over the years further irritates and inflames the synovium, resulting in severe joint pain and swelling, muscle weakness, impaired physical function, and limited joint movement. Chronic inflammation of synovium contributes significantly to synovitis, effusion, pain, chondrocyte apoptosis, progressive breakdown of the surface layer of articular cartilage, and eventually leads to osteoarthritis (OA) (Strobel, S., et al. (2010) Arthritis Res Ther 12, R34). Osteophytes may also grow on the edges of the joint, and bits of bone or cartilage can break off and float inside the joint, leading to further inflammation and damage. The prolonged inflammatory insult also increases the risk of arthrofibrosis for which there is no satisfactory management (Lotz, M. K., and Kraus, V. B. (2010) Arthritis Res Ther 12, 211). Accordingly, OA is a debilitating disease of the joint and is the most common degenerative joint disease, which afflicts about 50 million people with annual healthcare costs estimated to be 3 billion dollars in the U.S. in 2011 (Anderson, D. D., et al. (2011) J Orthop Res 29, 802-809). About 12% (~12.7 million) have had an acute trauma to the joint (Brown, T. D., Johnston, et al. (2006) J Orthop Trauma 20, 739-744). These OA are referred to as posttraumatic OA (PTOA). While primary OA affects mostly people older than 60 years of age, PTOA can afflict younger and more physically active individuals. The military population has a much greater incidence of PTOA than the general population due to intense military-related training and physical activities and to combat-related injuries to extremities (Cameron, K. L., et al. (2011) Arthritis Rheum 63, 2974-2982). OA/PTOA is also a major health issue for the Department of Veterans Affairs (VA), as veterans reported 50% higher incidence of OA than non-veterans and 43% of VA healthcare users had OA/PTOA (Dominick, K. L., et al. (2006) J Rheumatol 33, 348-354).

Damaged articular cartilage does not heal by itself. There is currently no cure for OA/PTOA. Patients with mild OA are treated with symptom-modifying medications limited to pain management and inflammation reduction (e.g., non-steroidal anti-inflammatory drugs (NSAIDs), duloxetine, physical therapy, cortisone, or hyaluronin) (Lotz., M. K., and Kraus, V. B. (2010) Arthritis Res Ther 12, 211), which are generally unsatisfactory (Matthews, G. L., and Hunter, D. J. (2011) Expert Opin Emerg Drugs 16, 479-491). Patients with severe OA may require surgical interventions that include marrow stimulation, structural osteo-articular autografts or autologous chondrocyte transplantation (Schindler, O. S. (2011) Acta Orthop Belg 77, 709-726), but these highly invasive surgical options are often disappointing. The frequent eventual endpoint is joint arthroplasty or even amputation. Joint arthroplasty is not a viable option for young or middle-aged patients, as it may need multiple revision surgeries over their lifetime to revise or replace failed implants (Anderson, D. D., et al. (2011) J Orthop Res 29, 802-809). Clinical trials focusing on pain alleviation (e.g., upadacitimib, tanezumab, Botox, UBX0101), anti-inflammatory/anti-catabolic activity aiming to suppress inflammation and further erosion of articular cartilage (e.g., AMG108, SM04690), and anabolic therapies assessing benefits of local injections of platelet-rich plasma and mesenchymal stem cells to regenerate the damaged articular cartilage are on-going. Many anti-catabolic therapeutic strategies, such as caspase inhibitors, matrix metalloproteinase (MMP) inhibitors, or anti-inflammatory cytokines (Lotz, M. K., and Kraus, V. B. (2010) Arthritis Res Ther 12, 211), anti-resorptive (Kadri, A., et al. (2010) Ann Rheum Dis 69, 1533-1538; and Shimizu, S., et al. (2007) Arthritis Rheum 56, 3358-3365), bone-sparing (Reginster, J. Y., et al. (2013) Ann Rheum Dis 72, 179-186; and Yano, F., et al. (2013) Ann Rheum Dis 72, 748-753), or chemokines and receptors (Scanzello, C. R. (2017) J Orthop Res 35, 735-739) or anabolic strategies involving chondrocyte-based, protein/scaffold-based, or gene transfer-based strategies to locally express a chondrogenic growth factor or transcription factor (Steinert, A. F., et al. (2008) Injury 39 Suppl 1, S97-113) are also at the various stages of preclinical development. While these strategies are effective in reducing certain symptoms, their efficacy is marginal and they fail to yield long-term benefits (Punzi, L., et al. (2016) RMD Open 2, e000279). Consequently, new and more effective therapies for OA and PTOA are badly needed.

### SUMMARY

Disclosed herein are compositions comprising, one or more liposomes, and a targeting peptide, wherein the one or more liposomes comprise a transmembrane protein, and wherein the targeting peptide is outside of the liposome.

Disclosed herein are compositions comprising, one or more osteoclast-derived microvesicles, and a targeting peptide, wherein the one or more osteoclast-derived microvesicles comprises a transmembrane protein, and wherein the targeting peptide is outside the one or more osteoclast-derived microvesicles.

Disclosed herein are compositions comprising, one or more osteoclast-derived microvesicles comprising EfnB2, and a targeting peptide, wherein the targeting peptide is outside of the one or more osteoclast-derived microvesicles.

Disclosed herein are methods of treating osteoarthritis or post-traumatic osteoarthritis in a subject, the methods comprising administering to the subject a therapeutically effective amount of a composition comprising, one or more liposomes, and a targeting peptide, wherein the one or more liposomes comprise a transmembrane protein, and wherein the targeting peptide is outside of the one or more liposomes.

Disclosed herein are methods of treating osteoarthritis or post-traumatic osteoarthritis in a subject, the methods comprising administering to the subject a therapeutically effective amount of a composition comprising, one or more osteoclast-derived microvesicles, and a targeting peptide, wherein the one or more osteoclast-derived microvesicles comprise a transmembrane protein, and wherein the targeting peptide is outside of the one or more osteoclast-derived microvesicles.

Disclosed herein are methods of upregulating chondrogenic gene expression in primary articular chondrocyte, the methods comprising administering to a subject a therapeutically effective amount of a composition comprising: (1) one or more liposomes, and a targeting peptide, wherein the one or more liposomes comprise a transmembrane protein, and wherein the targeting peptide is outside of the one or more liposomes or (2) one or more osteoclast-derived microvesicles, and a targeting peptide, wherein the one or more osteoclast-derived microvesicles comprises a transmembrane protein, and wherein the targeting peptide is outside the one or more osteoclast-derived microvesicles.

Disclosed herein are methods of regenerating articular cartilage and subchondral bone structure in a subject, the methods comprising administering to the subject a therapeutically effective amount of a composition comprising, one or more liposomes, and a targeting peptide, wherein the one or more liposomes comprise a transmembrane protein, and wherein the targeting peptide is outside of the one or more liposomes or one or more osteoclast-derived microvesicles, and a targeting peptide, wherein the one or more osteoclast-derived microvesicles comprise a transmembrane protein, and wherein the targeting peptide is outside the one or more osteoclast-derived microvesicles.

Disclosed herein are methods of promoting acidic proteoglycan-producing nodule formation in a subject, the methods comprising administering to the subject a therapeutically effective amount of a composition comprising, one or more liposomes, and a targeting peptide, wherein the one or more liposomes comprise a transmembrane protein, and wherein the targeting peptide is outside of the one or more liposomes or one or more osteoclast-derived microvesicles, and a targeting peptide, wherein the one or more osteoclast-derived microvesicles comprise a transmembrane protein, and wherein the targeting peptide is outside the one or more osteoclast-derived microvesicles.

Disclosed herein are methods of reducing or reversing synovial hyperplasia at an injured joint in a subject, the methods comprising administering to the subject a therapeutically effective amount of a composition comprising, one or more liposomes, and a targeting peptide, wherein the one or more liposomes comprise a transmembrane protein, and wherein the targeting peptide is outside of the one or more liposomes or one or more osteoclast-derived microvesicles, and a targeting peptide, wherein the one or more osteoclast-derived microvesicles comprise a transmembrane protein, and wherein the targeting peptide is outside the one or more osteoclast-derived microvesicles.

Disclosed herein are methods of reducing inflammation at an injured joint in a subject, the methods comprising administering to the subject a therapeutically effective amount of a composition comprising, one or more liposomes, and a targeting peptide, wherein the one or more liposomes comprise a transmembrane protein, and wherein the targeting peptide is outside of the one or more liposomes or one or more osteoclast-derived microvesicles, and a targeting peptide, wherein the one or more osteoclast-derived microvesicles comprise a transmembrane protein, and wherein the targeting peptide is outside the one or more osteoclast-derived microvesicles.

Disclosed herein are methods of promoting bone healing or increasing local bone formation, the methods comprising administering to a subject a therapeutically effective amount of a composition comprising, one or more liposomes, and a targeting peptide, wherein the one or more liposomes comprise a transmembrane protein, and wherein the targeting peptide is outside of the one or more liposomes or one or more osteoclast-derived microvesicles, and a targeting peptide, wherein the one or more osteoclast-derived microvesicles comprise a transmembrane protein, and wherein the targeting peptide is outside the one or more osteoclast-derived microvesicles.

### BRIEF DESCRIPTION OF THE DRAWINGS

FIGS. 1A-B show that osteoclast-derived MV upregulated the expression of chondrogenic genes (FIG. 1A) and enhanced formation of acidic proteoglycan-producing colonies (FIG. 1B) in primary articular chondrocytes. The osteoclast-derived MV was isolated by differential ultracentrifugation of conditioned media (CM) of RAW264.7 cells-derived osteoclasts (RAW-OC) after 6 days RANKL treatment. Primary chondrocytes were isolated from articular cartilage layers of C57BL/6 mice, cultured in DMEM/F12 containing 2% FBS, and incubated with an effective dose of the osteoclast-derived MV for 24 hr. FIG. 1A shows the relative expression level of chondrogenic genes that were determined by qRT-PCR using the ΔCT method and normalized against β-actin (mean±SEM, n=3). Dotted line represents each untreated control. P values are compared with each corresponding untreated control. FIG. 1B shows the acidic proteoglycan producing colonies. Alcian blue staining was eluted with 8M guanidine HCl from articular chondrocytes cultured in medium containing 2% FBS with PBS or RAW-OC MV for 21 days. Absorbance was read at 600 nm.

FIGS. 2A-B show that treatment of primary articular chondrocytes with osteoclastic MV, apoptotic bodies (AB), or exosomes (Exo) upregulated cellular expression of Pgr4 and increased the ability of formation of acidic proteoglycan-producing colonies in primary articular chondrocytes. FIG. 2A shows that while the test extracellular vesicles upregulated Pgr4 expression, osteoclastic MV showed the greatest enhancing activity. Cellular Pgr4 expression level was determined by RT-PCR (n=4). Control cells were treated with same volume of PBS. Results are shown as mean±SEM (n=4). P values are determined by two-tailed Student's t-test compared to respective control group. FIG. 2B shows that treatment with primary chondrocytes with osteoclastic MV, but not osteoclastic AB or Exo (at the same protein concentration), for 21 days increased the ability of primary chondrocytes to form acidic proteoglycan-producing colonies in culture medium containing 10% fetal calf serum in medium containing 10% FCS. Control cells were treated with PBS. P values are determined by one-way ANOVA compared to respective control group. The dashed line is 100% of the controls.

FIGS. 3A-B show that treatment of primary mouse synoviocytes with osteoclastic MV, AB, or Exo affects cellular expression levels of pro-inflammatory genes. FIG. 3A shows that osteoclastic MV downregulated the expression of several pro-inflammatory genes, e.g., Igf1, Cd44, pPBP, and Mmp9, in primary synoviocytes. Primary mouse synoviocytes were isolated from mouse knee joints (Zhao, J., et al. (2016) Biomed Rep 5, 171-175). Control cells were treated with PBS and experimental cells were treated with osteoclastic MV for 48 hours. Total RNA was extracted from control and experimental cells, and the expression levels of test genes were determined by RT-qPCR and normalized against eta-actin. Results are shown as mean±S.E.M., n=3. FIG. 3B shows that osteoclastic MV, AB, and Exo, each substantially suppressed the expression of genes associated with synovial inflammation in primary mouse synoviocytes. The relative cellular expression levels of synovial inflammation-associated genes, e.g., IGF1, CD44, pPBP, and Mmp9, in synoviocytes after incubation with the same protein concentration of MV, AB, or Exo (or PBS in control cells) for 48 hours. P values are determined by One-way ANOVA compared to respective control group. The dashed line is 100% of the controls. Results are shown as mean±S.E.M., n=3.

FIGS. 4A-E show that biweekly intraarticular injections of osteoclast-derived MV have beneficial effects on the prevention of OA. FIG. 4A shows toluidine blue staining (light gray) of acidic cartilage of a representative contralateral uninjured tibial joint and an injured joint of each group treated with PBS or osteoclast-derived MV. White arrows=areas with thinning of articular cartilage layer; gray arrows=osteophyte-like tissue. FIG. 4B shows the average thickness of articular cartilage on injured tibial joint, measured on multiple sections of each animal. *P<0.05, ***P<0.001, compared to PBS-treated injured joints. FIG. 4C shows the average area of osteophyte-like tissues on tibial joint measured on multiple sections of each animal of each test group. Results are shown as mean±SEM (n=4 mice) *P<0.05, ***P<0.001, compared to intact uninjured joints. FIG. 4D shows the average articular cartilage area on the injured tibial plateau site, measured on multiple sections of each mouse. Measurements of average articular cartilage area was performed with the OsteoMeasure system. Results are shown as mean±S.E.M., n=4 mice per group. Statistical analyses were performed with two-tailed Student's t-test. P values are compared to corresponding intact uninjured joint. FIG. 4E shows that osteoclastic MV treatment reduced the loss of articular cartilage surface smoothness and loss of gliding/intermediate zones induced by OA. Smoothness of articular cartilage surface and loss of gliding/intermediate zone area measurements were performed as described in Stiffel, V., (2020) Calcif Tissue Int 106, 158-171. Results are shown as mean±S.E.M., n=4 animals per group. Statistical significance was determined by two-tailed Student's t-test. P>0.05 is considered not significant.

FIG. 5 shows that biweekly intraarticular injections of osteoclast-derived MV reduced synovial hyperplasia after intraarticular tibial plateau injury. The arrows indicate the thickness of synovium. This figure shows that the intraarticular tibial plateau injury after 5 weeks substantially increased the thickness of the synovium at the injured joint, and the osteoclast-derived MV biweekly injections prevented or reduced the synovial hyperplasia.

FIG. 6 shows that biweekly intraarticular injections of osteoclastic MV reduced the severity of OA developed after the closed intraarticular tibial compression loading-induced injury at 5 weeks post-injury. The relative severity of OA was assessed with the 6 points-scale OA cartilage histopathology-based OARSI OA scoring system (Pritzker, K. P., et al. (2006) Osteoarthr Cartil 14, 13-29).

FIG. 7 shows a schematic representation of experimental strategy for comparison of single injection and multiple injections.

FIG. 8 Schematic representation of experimental strategy for testing efficacy of the disclosed osteoclast-derived MV-based strategy of administration of the therapy after OA/PTOA has already established.

FIG. 9 shows an experimental strategy for producing EfnB2-containing functionalized liposome-based microvesicles.

FIGS. 10A-B show microvesicles, but not apoptotic bodies (AB) or exosomes (Exo), released by RAW264.7 cells-derived osteoclasts (RAW-OC) stimulated bromouridine (BrdU) incorporation and cellular alkaline phosphatase (ALP) activity in primary mouse osteoblasts. FIG. 10A shows the BrdU incorporation reported as % control (mean±SEM, n=5-6). FIG. 10B shows the cellular ALP activity as mean±SEM, n=3. P values are determined by one-way ANOVA compared to the respective control group. Dashed lines indicate the 100% of corresponding controls.

FIGS. 11A-C show microvesicles isolated from pooled conditioned media (CM) of primary osteoclasts (OC), but not MC3T3-E1 or human embryonic kidney (HEK) cells, increased both BrdU incorporation (FIG. 11A) and cellular ALP activity (FIG. 11B) in MC3T3-E1 cells. MV of RAW264.7 cells-derived osteoclasts (RAW-OC) also promoted mineralized nodule formation after 21 days incubation (FIG. 11C) in primary osteoblasts. FIGS. 11A-B show that osteoclast-derived MV was added to MC3T3-E1 cells for 24 hrs (left) or 48 hrs (right). BrdU incorporation and ALP activity were each normalized against MV protein content. Mean±SEM, n=6. FIG. 11C shows the comparison of relative intensity of alizarin red staining indicates that MV of RAW-OC increased mineralized nodule formation by ~75%.

FIGS. 12A-B show that intracranial injections of osteoclast-derived microvesicles for 5 days increased local de novo bone formation at 14 days. FIG. 12A shows double tetracycline labeled surface (arrows) at the external layer. Bar=50 μm. FIG. 12B shows the dynamic histomorphometric bone formation parameters. Mean±SEM, n=3 per group.

FIGS. 13A-B show osteoclast-derived microvesicles upregulated the expression of chondrogenic genes (FIG. 13A) and formation of acidic proteoglycan-producing colonies (FIG. 10B) in primary chondrocytes. FIG. 13A shows the expression levels of chondrogenic genes determined by qRT-PCR and normalized against β-actin (mean±SEM, n=3). Dashed line=fold of each control value. FIG. 13B shows chondrocytes cultured in medium containing 2% FCS with PBS or RAW264.7 cells-derived osteoclastic (RAW-OC) MV for 21 days. Acidic proteoglycan producing colonies were stained with Alcian blue and eluted with 8M guanidine HCl (n=3). Absorbance was read at 600 nm. Dashed line=% of the controls.

FIG. 14 shows that osteoclast-derived microvesicles, but not apoptotic bodies (AB) or exosomes (Exo), significantly enhances the formation of acidic proteoglycan-producing colonies in primary chondrocytes. Acidic proteoglycan producing colonies formed in chondrocytes after incubation with AB, MV, and Exo of RAW264.7 cells-derived osteoclasts (RAW-OC) (at same protein concentration) or PBS (Control) in medium containing 10% FCS for 21 days. P values are determined by one-way ANOVA compared to respective control group. The dashed line is 100% of the controls. The level of stimulation by MV in this experiment is smaller than that shown in FIG. 13, because 10% FCS instead of 2% FCS was used.

FIGS. 15A-C show X-ray evidence that weekly injections of osteoclast-derived microvesicles into fractures promoted bony bridging of femoral fracture (FIG. 15A) and μCT reconstruction evidence support an enhanced bony callus formation (FIGS. 15B-C) at 21 or 28 days. FIG. 15A shows an X-ray photomicrograph of two healing fractures of the MV- or PBS-treated fractures at day 21 and 28 post-fracture. Arrows=fracture gaps. FIG. 15B shows μ-CT 3-D reconstruction of a PBS-treated and a RAW264.7 cells-derived (RAW-OC)-MV-treated fracture at 21 days. Left: external sagittal view, and Right: bisected sagittal view. The newly formed low-density (220-570 mg/cm3) woven bone is shown as dark gray to distinguish it from the native high-density (570-1000 mg/cm3) cortical bone. Scale=1 mm. FIG. 15C shows the quantitation of μCT parameters of woven bone at fracture calluses at 28 days. n=3 for PBS and n=2 for MV.

FIGS. 16A-B show that weekly osteoclast-derived microvesicle injections increased the amounts of Safranin O-stained cartilage in callus at day 14 post-fracture (FIG. 16A) and immunohistochemically stained vWF (as an index of neo-angiogenesis) at day 21 post-fracture (FIG. 16B). FIG. 16A shows cartilage stained in red. FIG. 16B shows vWF protein of newly formed bone vessels at fracture callus stained in brownish color.

FIG. 17 shows that the 24-hr treatment of mouse endothelial cells with osteoclast-derived microvesicles increased expression of endothelial cell maturation genes. Results are shown as fold of controls (Mean±SEM, n=3).

FIGS. 18A-B show that weekly injections of osteoclast-derived microvesicles increased ultimate torques (FIG. 18A) and torsional stiffness (FIG. 18B) of healing femoral fractures at 28 days compared to PBS-injected fractures. Results are shown as % of corresponding contralateral intact femur. Mean±SEM, n=6 for PBS group and n=4 for MV group in each panel. *P<0.05 and ***P<0.001 compared to intact femurs.

FIG. 19 shows the localization of EfnB2 with osteoclast-derived microvesicles fraction and that of rank with apoptotic bodies (AB). Osteoclast-derived MV and AB fractions were isolated from pooled conditioned media (CM) of RAW264.7 cell-derived osteoclasts (RAW-OC) by differential centrifugation. Sixty μg of osteoclast-derived MV or AB proteins were separated by SDS-PAGE and transblotted on nitrocellulose membrane. The same blot was immunoblotted against anti-EfnB2, and reblotted against anti-mouse Rank, anti-mouse Tsp1, and anti-mouse Arf6.

FIGS. 20A-B show that pretreatment of conditioned media (CM) of PTH-treated calvaria (FIG. 20A) or osteoclast-derived microvesicles (FIG. 20B) with an anti-EfnB2 antibody reduced its bone cell mitogen activity. FIG. 20A shows that the CM of calvaria treated with 10 nM PTH (to increase bone turnover) or vehicle was pre-treated with 100 μg/mL anti-EfnB2 antibodies or IgG for 2 h prior to the BrdU assay in MC3T3-E1 cells. FIG. 20B shows osteoclast-derived MV of primary osteoclasts pre-treated with 10 μg/mL anti-EfnB2 or IgG for 2 h before BrdU assay in MC3T3-E1 cells. Mean±SEM, n=4 per group.

FIG. 21 shows the shRNA-mediated knockdown of EfnB2 expression in RAW264.7 cell-derived osteoclasts (RAW-OC) increased TRAP activity (left) and reduced the microvesicles-mediated stimulation of ALP activity in MC3T3-E1 cells (right). Results are shown as % of MV of control lentivirus-treated RAW-OC, normalized against cellular TRAP. Mean±SEM, n=3 per group.

FIGS. 22-C show osteoclast-derived microvesicles of RAW264.7 cells-derived osteoclasts (RAW-OC) transduced with lenti-EfnB2-GFP induced increases in number (FIG. 22A) and cellular ALP activity (FIG. 22B) of MC3T3-E1 cells and enhanced mineralized nodule formation in primary osteoblasts (FIG. 22C) compared to MV of lenti-GFP-transduced RAW-OC. Same amount of protein of each MV was used. In FIG. 22C, EfnB2-overexpressing osteoclast-derived MV increased mineralized nodule formation by >2-fold compared to MV of lenti-GFP transduced RAW-OC. Mean±SEM, n=3.

FIGS. 23A-B shows that pretreatment of MC3T3-E1 cells with a neutralizing anti-EphB4 antibody reduced the ability of osteoclast-derived microvesicles to enhance BrdU incorporation (FIG. 23A) and cellular ALP activity (FIG. 23B). MC3T3-E1 cells were pretreated with 2 μg/mL anti-EphB4 antibodies or IgG for 1 h prior to addition of RAW264.7 cell-derived osteoclasts (RAW-OC) microvesicles and incubated for 24 hr. FIG. 23A shows that BrdU was added during the final 12 hr. Mean±SD (n=4). FIG. 23B shows that cellular ALP was measured after 48 hr incubation. Mean±SD, n=4.

FIG. 24 shows that treatment of chondrocytes with soluble EfnB2-fc (1 μg/mL) for 24 hr upregulated the expression of chondrogenic genes but not type I collagen. mRNA levels were determined by RT-qPCR and normalized against β-actin. Results are shown as folds of controls. Mean±SEM, n=3 per group.

FIGS. 25A-B show that weekly injections of EfnB2-deficient (EfnB2-Def) osteoclast-derived MV yielded less mineralized calluses than EfnB2-sufficient (EfnB2-Suff) osteoclast-derived MV at 14 days of healing of femoral fractures. EfnB2-deficient and EfnB2-sufficient osteoclast-derived MV were isolated from pooled control media (CM) of lenti-EfnB2 shRNA- or control lentivector-treated RAW264.7 cells-derived osteoclasts (RAW-OCs), respectively. The lenti-EfnB2 shRNA treatment reduced cellular EfnB2 mRNA by ~60% in RAW-OCs.

FIG. 25A is a representative PBS-, EfnB2-sufficient osteoclast-derived MV, and EfnB2-deficient osteoclast-derived MV-treated fracture each. Arrows=fracture site. Soft calluses of EfnB2-sufficient osteoclast-derived MV-treated fractures (n=5) appeared to have greater relative mineralization (as increased white shaded area within calluses) than those of EfnB2-deficient osteoclast-derived MV (n=4) and PBS (n=6) fractures. FIG. 25B shows the quantification of μCT parameters of newly formed low-density (220-570 mg/cm³) woven bone in soft callus of the three fracture groups.

FIG. 26 shows the proposed molecular mechanism of the soluble osteoclast-derived microvesicles regulatory system of local bone formation/regeneration.

FIG. 27 shows the strategy that will be used to compare efficacy of single vs multiple injections of osteoclast-derived microvesicles in enhancing callus cartilage formation and remodeling.

FIG. 28 shows the strategy that will be used to confirm beneficial effects of osteoclast-derived microvesicles on the return of bone strength.

### DETAILED DESCRIPTION

The present disclosure can be understood more readily by reference to the following detailed description of the invention, the figures and the examples included herein.

Before the present methods and gene expression panels are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g., a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment prior to the administering step.

As used herein, the term “comprising” can include the aspects “consisting of” and “consisting essentially of.”

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with osteoarthritis or post-traumatic osteoarthritis” or “diagnosed with having had or at risk for having osteoarthritis or post-traumatic osteoarthritis” means having been subjected to a physical examination by a person of skill, for example, a physician, and found to have a condition that can be diagnosed or can be treated by a composition that can upregulate chondrogenic gene expression in primary articular chondrocytes, regenerate articular cartilage and subchondral bone structure, promote acidic proteoglycan-producing nodule formation, reduce or reverse synovial hyperplasia at an injured joint, reduce inflammation at an injured joint, reducing one or more cytokines (e.g., IL-1β, IL-6, TNFα, IL-2, IL-7, IL-15, and IL-21) in the joint, promote bone healing or increase local bone formation or a combination thereof.

As used herein, the phrase “identified to be in need of treatment for osteoarthritis or post-traumatic osteoarthritis,” or the like, refers to selection of a subject based upon need for treatment of the osteoarthritis or post-traumatic osteoarthritis. For example, a subject can be identified as having a need for treatment of osteoarthritis or post-traumatic osteoarthritis (based upon an earlier diagnosis by a person of skill and thereafter subjected to treatment for osteoarthritis or post-traumatic osteoarthritis). It is contemplated that the identification can, in some aspects, be performed by a person different from the person making the diagnosis. It is also contemplated, in a further aspect, that the administration can be performed by one who performed the diagnosis.

“Inhibit,” “inhibiting” and “inhibition” mean to diminish or decrease or reduce an activity, level, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in an aspect, the inhibition or reduction can be a 10, 20, 30, 40, 50, 60, 70, 80, 90, 100%, or any amount of reduction in between as compared to native or control levels. In an aspect, the inhibition or reduction is 10-20, 20-30, 30-40, 40-50, 50-60, 60-70, 70-80, 80-90, or 90-100% as compared to native or control levels. In an aspect, the inhibition or reduction is 0-25, 25-50, 50-75, or 75-100% as compared to native or control levels.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in some aspects, the increase or promotion can be a 10, 20, 30, 40, 50, 60, 70, 80, 90, 100%, or more, or any amount of promotion in between compared to native or control levels. In some aspects, the increase or promotion is 10-20, 20-30, 30-40, 40-50, 50-60, 60-70, 70-80, 80-90, or 90-100% as compared to native or control levels. In some aspects, the increase or promotion is 0-25, 25-50, 50-75, or 75-100%, or more, such as 200, 300, 500, or 1000% more as compared to native or control levels. In some aspects, the increase or promotion can be greater than 100 percent as compared to native or control levels, such as 100, 150, 200, 250, 300, 350, 400, 450, 500% or more as compared to the native or control levels.

As used herein the term “effective amount” or “therapeutically effective amount” can refer to an amount of agent, such as compositions comprising any of the liposomes, compositions or pharmaceutical compositions described herein, that is sufficient to generate a desired response, such as reducing or eliminating a sign or symptom of a condition or disease, such as a osteoarthritis or post-traumatic osteoarthritis. In some aspects, the condition or disease can be characterized by downregulation of chondrogenic gene expression in primary articular chondrocytes. Such signs or symptoms can include upregulation of chondrogenic gene expression in primary articular chondrocytes, regenerate articular cartilage and subchondral bone structure, promote acidic proteoglycan-producing nodule formation, reduce or reverse synovial hyperplasia at an injured joint, reduce inflammation at an injured joint, reducing one or more cytokines (e.g., IL-1β, IL-6, TNFα, IL-2, IL-7, IL-15, and IL-21) in the joint, promote bone healing or increase local bone formation or a combination thereof.

Also, as used herein, the terms “effective amount”, “amount effective”, and “therapeutically effective amount” can refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. For example, in some aspects, an effective amount of a disclosed liposome, composition or pharmaceutical composition is the amount effective upregulate chondrogenic gene expression in primary articular chondrocytes, regenerate articular cartilage and subchondral bone structure, promote acidic proteoglycan-producing nodule formation, reduce or reverse synovial hyperplasia at an injured joint, reduce inflammation at an injured joint, reducing one or more cytokines (e.g., IL-1β, IL-6, TNFα, IL-2, IL-7, IL-15, and IL-21) in the joint, promote bone healing or increase local bone formation or a combination thereof. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a disclosed composition at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. In some aspects, a preparation can be administered in a “prophylactically effective amount”; that is, an amount effective for prevention of a disease or condition.

As used herein, “increase” or “upregulation” of chondrogenic gene expression in primary articular chondrocytes refers to the production of a gene product or protein in a subject or a sample that exceeds levels of production in normal, control, or non-diseased subject.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described.

Publications cited herein and the materials for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert and applicants reserve the right to challenge the accuracy and pertinency of the cited documents. It will be clearly understood that, although a number of publications are referred to herein, such reference does not constitute an admission that any of these documents forms part of the common general knowledge in the art.

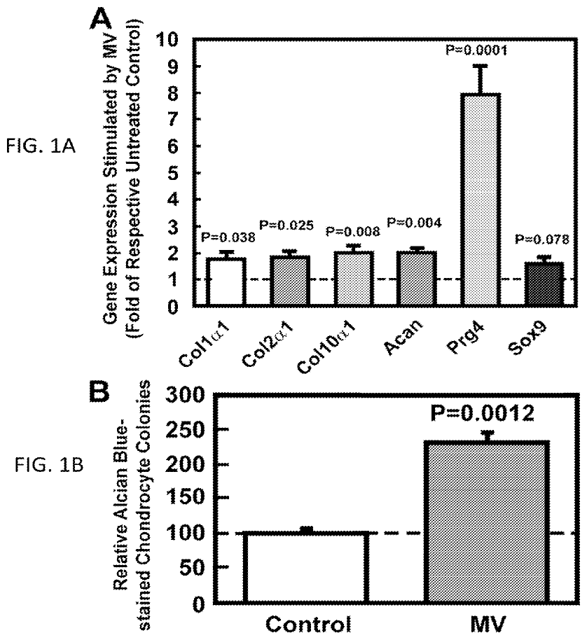

Osteoclast-derived microvesicles (MV) have osteogenic and chondrogenic activities. MV is formed by budding directly from plasma membrane of living cells (Heijnen, H. F., et al. (1999) Blood 94, 3791-3799) and is a major form of extracellular vesicles (EV). EV are nanosized, membrane-bound vesicles released by cells that can transport DNA, RNA, or protein biologic cargoes between distant cells as a form of intercellular communication (Balaj, L., et al. (2011) Nat Commun 2, 180). Described herein are microvesicle-based (MV based) strategies that can be used to deliver, for example, EfnB2.EV-based strategy is an emerging technology for delivering therapeutics, genes, or vaccines (Lakhal, S., and Wood, M. J. (2011) BioEssays: news and reviews in molecular, cellular and developmental biology 33, 737-741) Apoptotic osteoclasts release osteoanabolic apoptotic bodies (AB) that may play a role regulating the local bone formation response to an increase in bone resorption (Liu, M., et al. (2018) Journal of dental research 97, 859-868). Also disclosed herein are results showing that the osteoclastic regulation of local coupled bone formation MV derived from osteoclasts can be used as a therapeutic agent for OA/PTOA having both pro-chondrogenic and osteogenic effects. Accordingly, the results disclosed herein show that a) living osteoclasts released predominantly osteoanabolic MV to regulate local bone formation; b) MV derived from osteoclasts but not osteoblastic cells showed potent stimulatory activities on the proliferation, differentiation, and mineralized nodule formation in osteoprogenitor cells; c) direct injections of osteoclast-derived MV at the calvarium increased local de novo bone formation at outer surface of the injected calvarium in vivo; and d) osteoclast-derived MV upregulated chondrogenic gene expression and promoted acidic proteoglycan-producing nodule formation in primary articular chondrocytes (FIG. 1), indicative of an pro-chondrogenic effect. The in vivo pro-chondrogenic activity of osteoclast-derived MV is further exemplified by the finding that injections of osteoclast-derived MV into the fracture site enhanced endochondral healing of femoral fractures. Because persistent and progressive erosion of articular cartilage, periarticular and subchondral bones are pathologic hallmarks of OA, an agent, such as osteoclast-derived MV, that promotes regeneration of articular cartilage and subchondral bone structure can be an attractive candidate for a comprehensive therapy for OA/PTOA.

Described herein is the use of microvesicle (MV), a physiological cell product, as the therapeutic for OA for treatment of OA or related joint disease. The use of a physiological product, such as MV, would have an additional theoretical benefit in that it would not cause appreciable immune or inflammation responses. Thus, it is expected that the MV-based strategy would have a favorable safety profile. Osteoclast-derived MVs have multiple biological activities that are relevant to treatment of OA, such as pro-chondrogenic, anti-inflammatory, and osteogenic activities. Thus, osteoclast-derived MV can be used as a therapy for OA because it addresses multiple important pathologies that would allow for a more comprehensive treatment. Importantly, this strategy differs from the currently available therapies, which address a single pathology of OA (either anti-catabolic or pro-anabolic, but not both). Also described herein is the use of cells of monocytes/macrophage lineage to produce the MV, thereby allowing the use of the patient's own blood as the cell source of osteoclast-derived MV, which is clinically beneficial. MV is also stable in circulation and in body fluids, as the membrane structure of MV would protect itself from being cleared or degraded by circulating proteases. The MV-based strategy described herein would have a relatively long biological half-life, which then leads to prolonged potency.

### Compositions

Disclosed herein are compositions comprising one or more liposomes and a targeting peptide. In some aspects, the one or more liposomes can comprise a transmembrane protein. In some aspects, the targeting peptide can be outside of the liposome. Disclosed herein are compositions comprising, one or more liposomes, and a targeting peptide, wherein the one or more liposomes comprise a transmembrane protein, and wherein the targeting peptide is outside one or more of the liposomes.

Also disclosed herein are compositions comprising, one or more osteoclast-derived microvesicles, and a targeting peptide. In some aspects, the one or more osteoclast-derived microvesicles can comprise a transmembrane protein. In some aspects, the targeting peptide can be outside the one or more osteoclast-derived microvesicles. Disclosed herein are compositions comprising, one or more osteoclast-derived microvesicles, and a targeting peptide, wherein the one or more osteoclast-derived microvesicles comprise a transmembrane protein, and wherein the targeting peptide is outside one or more of the osteoclast-derived microvesicles.

In some aspects, the one or more liposomes can be a multilamellar vesicle. In some aspects, the multilamellar vesicle can comprise 1,2-dioleoyl-3-trimethylammonium-propane (DOTAP), Dioleoylphosphatidylethanolamine (DOPE), cholesterol, DSPE-mPEG2000, DSPE-PEG2000-MAL, or a combination thereof. In some aspects, the multilamellar vesicle comprises 1,2-dioleoyl-3-trimethylammonium-propane (DOTAP), Dioleoylphosphatidylethanolamine (DOPE), cholesterol, DSPE-mPEG2000, and DSPE-PEG2000-MAL. In some aspects, the multilamellar vesicle comprises 1,2-dioleoyl-3-trimethylammonium-propane (DOTAP), Dioleoylphosphatidylethanolamine (DOPE), cholesterol, DSPE-mPEG2000, and DSPE-PEG2000-MAL in a molar ratio of 42:15:38:3:2, respectively. In some aspects, the molar concentration of the DOTAP can be 50 μM, 75 μM, 150 μM, 250 μM, or 500 μM, or any number in between, the DOPE can be 50 μM, 75 μM, 150 μM, 250 μM, or 500 μM, or any number in between the cholesterol can be 50 μM, 125 μM, 250 μM, 500 μM, or 1 mM, or any number in between, the DSPE-mPEG2000 can 50 μM, 75 μM, 150 μM, 250 μM, or 500 μM, or any number in between, and the DSPE-PEG2000-MAL can 50 μM, 75 μM, 150 μM, 250 μM, or 500 μM, or any number in between.

A microvesicle (MV) is a form of an extracellular vesicles (EV). As disclosed herein, MVs can be used as an intracellular biological vehicle to deliver various biological (e.g., nucleic acids, protein, peptide, and non-peptide) agents. The major forms of EVs include MVs, apoptotic bodies, exosomes, and oncosomes. Exosomes and apoptotic bodies have bone forming activity. As disclosed herein, MVs derived from osteoclasts have potent bone regeneration activities. In some aspects, the active component in an osteoclastic MV can be EfnB2.

In some aspects, the transmembrane protein of the claimed compositions can be EfnB2, an EfnB2 isoform, or a modified EfnB2. In some aspects, a modified EfnB2 can be a recombinant form of EfnB2. In some aspects, the targeting peptide can be added to the N-terminus of the transmembrane protein. For example, an expression construct can be generated by adding the targeting peptide (e.g., SEQ ID NO: 1) to the N-terminus of transmembrane protein (e.g., EfnB2). Next, osteoclast precursor cells can be infected with lentiviral or other viral or nonviral transfection vectors to express this modified or recombinant EfnB2. The infected osteoclast precursors can be converted with RANKL/m-CSF to form functional osteoclasts and then MV can be isolated from these osteoclasts to provide osteoclast-derived MV containing the modified or recombinant EfnB2. In some aspects, expression constructs can be used to produce recombinant or modified EfnB2 that can be used to produce liposomes that contain the modified or recombinant EfnB2. In some aspects, the transmembrane protein can be overexpressed in the osteoclast-derived MV or liposome.

In some aspects, EfnB2 can be a human EfnB2 isoform. In some aspects, human EFNB2 isoform 1 precursor has the protein sequence corresponding to Accession No: NP_004084, and the amino acid sequence of:

(SEQ ID NO: 2)
MAVRRDSVWKYCWGVLMVLCRTAISKSIVLEPIYWNSSNSKFLPGQGLV
LYPQIGDKLDIICPKVDSKTVGQYEYYKVYMVDKDQADRCTIKKENTPL
LNCAKPDQDIKFTIKFQEFSPNLWGLEFQKNKDYYIISTSNGSLEGLDN
QEGGVCQTRAMKILMKVGQDASSAGSTRNKDPTRRPELEAGTNGRSSTT
SPFVKPNPGSSTDGNSAGHSGNNILGSEVALFAGIASGCIIFIVIIITL
VVLLLKYRRRHRKHSPQHTTTLSLSTLATPKRSGNNNGSEPSDIIIPLR
TADSVFCPHYEKVSGDYGHPVYIVQEMPPQSPANIYYKV.

In some aspects, human EfnB2 Isoform 2 has the protein sequence corresponding to Accession No: NP_001358985.1, and the amino acid sequence of:

(SEQ ID NO: 3)
MAVRRDSVWKYCWGVLMVLCRTAISKSIVLEPIYWNSSNSKFLPGQGLV
LYPQIGDKLDIICPKVDSKTVGQYEYYKVYMVDKDQADRCTIKKENTPL
LNCAKPDQDIKFTIKFQEFSPNLWGLEFQKNKDYYIIYASSAGSTRNKD
PTRRPELEAGTNGRSSTTSPFVKPNPGSSTDGNSAGHSGNNILGSEVAL
FAGIASGCIIFIVIIITLVVLLLKYRRRHRKHSPQHTTTLSLSTLATPK
RSGNNNGSEPSDIIIPLRTADSVFCPHYEKVSGDYGHPVYIVQEMPPQS
PANIYYKV.

In some aspects, human EfnB2 Isoform 3 has the protein sequence corresponding to Accession No: NP_001358986, and the amino acid sequence of:

(SEQ ID NO: 4)
MAVRRDSVWKYCWGVLMVLCRTAISKSIVLEPIYWNSSNSKFLPGQGLV
LYPQIGDKLDIICPKVDSKTVGQYEYYKVYMVDKDQADRCTIKKENTPL
LNCAKPDQDIKFTIKFQEFSPNLWGLEFQKNKDYYIISTSNGSLEGLDN
QEGGVCQTRAMKILMKVGQGSSTDGNSAGHSGNNILGSEVALFAGIASG
CIIFIVIIITLVVLLLKYRRRHRKHSPQHTTTLSLSTLATPKRSGNNNG
SEPSDIIIPLRTADSVFCPHYEKVSGDYGHPVYIVQEMPPQSPANIYYK
V.

In some aspects, human EfnB2 protein has the protein sequence corresponding to Accession No: P52799, and the amino acid sequence of:

(SEQ ID NO: 5)
MAVRRDSVWKYCWGVLMVLCRTAISKSIVLEPIYWNSSNSKFLPGQGLV
LYPQIGDKLDIICPKVDSKTVGQYEYYKVYMVDKDQADRCTIKKENTPL
LNCAKPDQDIKFTIKFQEFSPNLWGLEFQKNKDYYIISTSNGSLEGLDN
QEGGVCQTRAMKILMKVGQDASSAGSTRNKDPTRRPELEAGTNGRSSTT
SPFVKPNPGSSTDGNSAGHSGNNILGSEVALFAGIASGCIIFIVIIITL
VVLLLKYRRRHRKHSPQHTTTLSLSTLATPKRSGNNNGSEPSDIIIPL
RTADSVFCPHYEKVSGDYGHPVYIVQEMPPQSPANIYYKV.

In some aspects, the transmembrane protein of the disclosed compositions can be a fragment of EfnB2. In some aspects, the fragment of EfnB2 can be a dimer. Accordingly, synthetic soluble forms of EfnB2 use the constant domain of IgG to conjugate with the extracellular domain of EfnB2 to form EfnB2-fc chimeric protein (e.g., the fc motif would aid their dimerization when pre-treated with IgG). In some aspects, the soluble extracellular domain of EfnB2 is capable of binding its receptor (e.g., EphB4) but is unable to activate the signaling mechanism of EphB4, and thereby is shown to be a potent inhibitor of full length EfnB2.

Any of the major isoforms for human EfnB2 protein can be used in the compositions disclosed herein. In some aspects, amino acid residues 1-27 of SEQ ID NOs: 2-5 are the signal peptide that can be replaced with, for example, the targeting peptide (e.g., the DSS6 peptide (SEQ ID NO: 1) for bone targeting. In some aspects, amino acid residues 28-229 of SEQ ID NOs: 2-4 are the extracellular domain, and are important for binding and activating the EphB4 receptor. In some aspects, amino acid residues 230-250 of SEQ ID NOs: 2-4 are at the transmembrane domain. In some aspects, amino acid residues 251 and beyond are intracellular domain, and can be replaced with other domains, such as, for example, the constant domain of IgG to produce the EfnB2-fc chimeric protein that can be used to produce soluble EfnB2.

In some aspects, the targeting peptide of the disclosed compositions can comprise or consist of DSS DSS DSS DSS DSS DSS (SEQ ID NO: 1). In some aspects, the targeting peptide can comprise or consist of DSS DSS DSS DSS DSS DSS (SEQ ID NO: 1) binding to hydroxyapaptide crystals.

In some aspects, the targeting peptide of the disclosed compositions can be a bone-targeting peptide. In some aspects, the targeting peptide can be of the hydroxyapaptide class. Examples of hydroxyapaptide targeting peptides include but are not limited to poly(Asp) (e.g., (Asp)8 (SEQ ID NO: 6) or (Asp)14 (SEQ ID NO: 7); (AspSerSer)6 (SEQ ID NO: 8); and HA-pep3 (e.g., LysAsnPheGlnSerArgSerHis (SEQ ID NO: 9). In some aspects, the targeting peptide can be of the osteoblast specific factor-2 class. Examples of osteoblast specific factor-2 targeting peptides include but are not limited to Ser-Asp-Ser-Ser-Asp (SEQ ID NO: 10).

The following references teach targeting peptides that can be useful in the disclosed invention and are incorporated herein by reference: Mol Therapy: Methods & Clin Dev (2020) 17 (Doi: 10.1016/j.omtm.2020.04.010); J Bone Miner Res (2000) 15:936-943 (doi: 10.1359/jbmr.2000.15.5.936); Lancet (2004) 363:787-787 (doi: 1016/S0140-6736 (04) 15717-6); Bone (2010) 47: S413-S414 (doi: 10.1016/j.bone.2010.09.216); SciRep (2020) 10:10576 (doi: 10.1038/S41598-020-67522-4); and ACS Nano (2016) (10:5759-5768 doi: 10.1021/acsnano.b07828).

### Pharmaceutical Compositions

As disclosed herein, are pharmaceutical compositions comprising any of the liposomes or osteoclast-derived microvesicles described herein or any of the compositions described herein. In some aspects, the pharmaceutical compositions disclosed herein can further comprise an aqueous solution. In some aspects, the aqueous solution can comprise the liposomes or osteoclast-derived microvesicles. In some aspects, the aqueous solution of the pharmaceutical composition can be adjusted to a human physiological pH. In some aspects, the pharmaceutical composition can be formulated for local administration. In some aspects, the pharmaceutical composition can be formulated for intraarticular administration. In some aspects, the pharmaceutical composition can be formulated for intravenous, oral, subcutaneous, intramuscular, or intranasal administration. The compositions of the present disclosure also contain a therapeutically effective amount of any of the liposomes, osteoclast-derived microvesicles, or compositions as described herein. The compositions can be formulated for administration by any of a variety of routes of administration, and can include one or more physiologically or pharmaceutically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The pharmaceutical compositions disclosed herein can be prepared for intra-articular, oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used to deliver the liposomes. Thus, compositions can be prepared for parenteral administration that includes liposomes suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like). Where the compositions are formulated for application to the skin or to a mucosal surface, one or more of the excipients can be a solvent or emulsifier for the formulation of a cream, an ointment, and the like.

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. In some aspects, a dry pharmaceutical composition can be formed by drying the pharmaceutical composition. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

In some aspects, the disclosed pharmaceutical composition or any of the compositions disclosed herein or any of the liposomes disclosed herein or any of the osteoclast-derived microvesicles disclosed herein can be administered in combination with one or more therapeutic agents. In some aspects, the one or more therapeutic agents can be used to treat any type of osteoarthritis Dosing with any of the one or more therapeutic agents (and other pharmaceutical compositions) is known in the art and can be altered to be used in combination with the disclosed pharmaceutical compositions or any of the compositions disclosed herein or any of the liposomes disclosed herein or any of the osteoclast-derived microvesicles disclosed herein by one of skill in the art in light of this disclosure. The one or more therapeutic agents can be given concurrently with, prior to or after the administration of any of the disclosed pharmaceutical compositions, or any of the compositions disclosed herein or any of the liposomes disclosed herein or any of the osteoclast-derived microvesicles disclosed herein.

### Methods of Treatment

Disclosed herein are methods of treating osteoarthritis or post-traumatic osteoarthritis, in a subject. In some aspects, the methods can comprise administering to the subject with osteoarthritis or post-traumatic osteoarthritis a therapeutically effective amount of any of the liposomes, osteoclast-derived microvesicles, pharmaceutical compositions, or compositions disclosed herein.

Disclosed herein are methods of ameliorating one or more symptoms of osteoarthritis or post-traumatic osteoarthritis. In some aspects, the methods can comprise administering to a subject with osteoarthritis or post-traumatic osteoarthritis a therapeutically effective amount of any of the liposomes, osteoclast-derived microvesicles, pharmaceutical compositions or compositions disclosed herein.

Disclosed herein are methods of upregulating chondrogenic gene expression in primary articular chondrocytes. In some aspects, the methods can comprise administering to a subject a therapeutically effective amount of any of the liposomes, osteoclast-derived microvesicles, pharmaceutical compositions, or compositions disclosed herein.

Disclosed herein are methods of regenerating articular cartilage and subchondral bone structure in a subject. In some aspects, the methods can comprise administering to the subject a therapeutically effective amount of any of the liposomes, osteoclast-derived microvesicles, pharmaceutical compositions, or compositions disclosed herein.

Disclosed herein are methods of promoting acidic proteoglycan-producing nodule formation in a subject. In some aspects, the methods can comprise administering to the subject a therapeutically effective amount of any of the liposomes, osteoclast-derived microvesicles, pharmaceutical compositions, or compositions disclosed herein.

Disclosed herein are methods of regenerating articular cartilage and subchondral bone structure in a subject. In some aspects, the methods can comprise administering to the subject a therapeutically effective amount of any of the liposomes, osteoclast-derived microvesicles, pharmaceutical compositions, or compositions disclosed herein.

Disclosed herein are methods of reducing or reversing synovial hyperplasia at an injured joint in a subject. In some aspects, the methods can comprise administering to the subject a therapeutically effective amount of any of the liposomes, osteoclast-derived microvesicles, pharmaceutical compositions, or compositions disclosed herein. In some aspects, the injured joint can be a hand (e.g., knuckle), lower back, neck, and weight-bearing joint (e.g., knee, hip, ankle, or joint in the feet).

Disclosed herein are methods of reducing inflammation at an injured joint in a subject. In some aspects, the methods can comprise administering to the subject a therapeutically effective amount of any of the liposomes, osteoclast-derived microvesicles, pharmaceutical compositions, or compositions disclosed herein. In aspects, the injured joint can be a hand (e.g., knuckle), lower back, neck, and weight-bearing joint (e.g., knee, hip, ankle, or joint in the feet). In some aspects, administering to the subject a therapeutically effective amount of any of the liposomes, osteoclast-derived microvesicles, pharmaceutical compositions, or compositions disclosed herein can reduce one or more cytokines in the joint of the subject. In some aspects, the one or more cytokines can be IL-1β, IL-6, TNFα, IL-2, IL-7, IL-15, or IL-21.

Disclosed herein are methods of promoting bone healing or increasing local bone formation. In some aspects, the methods can comprise administering to a subject a therapeutically effective amount of any of the liposomes, osteoclast-derived microvesicles, pharmaceutical compositions, or compositions disclosed herein.

Disclosed herein are methods of treating osteolytic bone lesions in a subject. In some aspects, the methods can comprise administering to the subject a therapeutically effective amount of any of the liposomes, osteoclast-derived microvesicles, pharmaceutical compositions, or compositions disclosed herein.

Disclosed herein are methods of enhancing the healing of bone fractures or defects in a subject. In some aspects, the methods can comprise administering to the subject a therapeutically effective amount of any of the liposomes, osteoclast-derived microvesicles, pharmaceutical compositions, or compositions disclosed herein. In some aspects, the bone defect can be a segmental bone defect. In some aspects, the bone defect can be a subsegmental bone defect. In some aspects, the bone defect can be a mandibular bone defect. In some aspects, the bone defect can be a calvarial bone defect. In some aspects, the bone fracture can be in long bones, including but not limited to a leg, an arm, a hand, a wrist, or a foot. In some aspects, the bone fractures can be in the vertebra. In some aspects, the bone fractures can be in a hip. In some aspects, the liposomes, osteoclast-derived microvesicles, pharmaceutical compositions, or compositions disclosed herein can be used in bone grafting. Thus, disclosed herein, are methods of repairing bone defects in a subject. In some aspects, the methods can comprise administering to the subject a therapeutically effective amount of any of the liposomes, osteoclast-derived microvesicles, pharmaceutical compositions, or compositions disclosed herein.

Disclosed herein are methods of treating osteoporosis in a subject. In some aspects, the methods can comprise administering to the subject a therapeutically effective amount of any of the liposomes, osteoclast-derived microvesicles, pharmaceutical compositions, or compositions disclosed herein. In some aspects, the liposomes, osteoclast-derived microvesicles, pharmaceutical compositions, or compositions can be locally administered in the hip, vertebral, long bones, and/or wrist fracture sites.

In some aspects, the subject can be identified in need of treatment before the administration step. In some aspects, the subject can be identified as having osteoarthritis or post-traumatic osteoarthritis in one or more injured joints. Osteoarthritis can be any type of osteoarthritis. For example, there are two types of osteoarthritis: primary and secondary osteoarthritis. Osteoarthritis is generally referred to as primary osteoarthritis. Post-traumatic osteoarthritis is considered secondary osteoarthritis that is caused by injuries to the joints. Other examples of osteoarthritis can include but is not limited to osteoarthritis that can develop in postmenopausal osteoporotic women, sometimes referred to as postmenopausal osteoarthritis. Examples of osteoarthritis include but are not limited to hip osteoarthritis (e.g., degenerative arthritis of the hip), spinal osteoarthritis (e.g., degenerative arthritis of the spine), shoulder osteoarthritis (e.g., degenerative arthritis of the shoulder), and cervical osteoarthritis (e.g., cervical spondylosis). In some aspects, the subject can have a skeletal fracture or bone defect. In some aspects, the subject can have large segmental bone defect. In some aspects, the subject can have one or more bone fractures. In some aspects, the subject can have one or more bone injuries. In some aspects, the injured joint can be a hand (e.g., knuckle), lower back, neck, and weight-bearing joint (e.g., knee, hip, ankle, or joint in the feet). In some aspects, the injured joint can be the shoulder or elbow. In some aspects, the subject can be a human. In some aspects, the subject can be identified as having osteoporosis. In some aspects, osteoporosis can be primary osteoporosis. In some aspects, primary osteoporosis can occur in the hand, foot, ankle, toe(s), and/or finger(s).

The pharmaceutical compositions described above can be formulated to include a therapeutically effective amount of any of the liposomes, osteoclast-derived microvesicles, or compositions disclosed herein. Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to any type of osteoarthritis or post-traumatic osteoarthritis.

The pharmaceutical compositions described herein can be administered to the subject (e.g., a human patient) in an amount sufficient to delay, reduce, or preferably prevent the onset of clinical disease. Accordingly, in some aspects, the patient can be a human patient. In therapeutic applications, compositions can be administered to a subject (e.g., a human patient) already with or diagnosed with any type of osteoarthritis or post-traumatic osteoarthritis in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a pharmaceutical composition can be an amount that achieves a cure, but that outcome is only one among several that can be achieved. As noted, a therapeutically effective amount includes amounts that provide a treatment in which the onset or progression of the osteoarthritis or post-traumatic osteoarthritis is delayed, hindered, or prevented, or the osteoarthritis or post-traumatic osteoarthritis or a symptom of the osteoarthritis or post-traumatic osteoarthritis is ameliorated. One or more of the symptoms can be less severe. Recovery can be accelerated in an individual who has been treated.

Amounts effective for this use can depend on the severity of the osteoarthritis or post-traumatic osteoarthritis and the weight and general state and health of the subject, but generally range from about 0.05 μg to about 1000 μg (e.g., 0.5-1000 μg) per kg body weight of an equivalent amount of the liposome or composition per dose per subject. Suitable regimes for initial administration and booster administrations are typified by an initial administration followed by repeated doses at one or more hourly, daily, weekly, biweekly, or monthly intervals by a subsequent administration. For example, a subject can receive any of the pharmaceutical compositions, compositions, osteoclast-derived microvesicles, or liposomes one or more times per week (e.g., 2, 3, 4, 5, 6, or 7 or more times per week). For example, a subject may receive a dose of 0.1 to 2,500 μg (e.g., 2,000, 1,500, 1,000, 500, 100, 10, 5, 1, 0.5, or 0.1 μg) per kg body weight per week. A subject can also receive a liposomes or compositions in the range of 0.1 to 3,000 μg per kg body weight per dose once every two or three weeks. A subject can also receive 2 mg/kg body weight every week (with the weight calculated based on the weight of the liposomes, osteoclast-derived microvesicles, or compositions).

The total effective amount of a liposomes, osteoclast-derived microvesicles, compositions or pharmaceutical compositions disclosed herein can be administered to a mammal as a single dose, either as a bolus or by infusion over a relatively short period of time, or can be administered using a fractionated treatment protocol in which multiple doses are administered over a more prolonged period of time (e.g., a dose every 4-6, 8-12, 14-16, or 18- 24 hours, or every 2-4 days, 1-2 weeks, or once a month). In some aspects, the liposomes, osteoclast-derived microvesicles, compositions or pharmaceutical compositions disclosed herein can be administered by local injections.

The therapeutically effective amount of one or more of the therapeutic agents present within the compositions described herein and used in the methods as disclosed herein applied to mammals (e.g., humans) can be determined by one of ordinary skill in the art with consideration of individual differences in age, weight, and other general conditions (as mentioned above).

### EXAMPLES

### Example 1: Osteoclast-Derived MV has Anabolic Actions on Articular Chondrocytes and Osteoprogenitors and Anti-Inflammatory Activity on Synoviocytes

To test whether osteoclast-derived MV has anabolic actions on articular chondrocyte and osteoprogenitors and anti-inflammatory activity on synoviocytes: a) it will be established that osteoclastic MV has pro-chondrogenic, osteogenic, and anti-inflammatory activities on articular chondrocytes, osteoprogenitors, and synoviocytes, respectively; b) it will be determined if these biological activities are specific to the MV subfraction, but not to other EV subfractions, of osteoclasts by comparing the pro-chondrogenic, osteogenic, and anti-inflammatory activities of MV with those of AB and exosomes (Exo) isolated from the same CM of osteoclasts; and c) it will be determined if the biological activities are specific to MV released by both differentiated and undifferentiated osteoclasts, but not osteoblasts, articular chondrocytes, or synoviocytes.

Experimental strategies. Experimental strategy 1a, has three sets of experiments, and focuses on establishing the anabolic effects of osteoclastic MV on articular chondrocytes and osteoprogenitors as well as anti-inflammatory action on synoviocytes in vitro. The first set of experiments is to establish the pro-chondrogenic effects of osteoclastic MV on the proliferation, apoptosis, maturation, and functional activity of primary articular chondrocytes (isolated according to established procedure (Stiffel, V. M., et al. (2020) Calcif Tissue Int 107, 576-592). Chondrocyte apoptosis will be measured because chondrocyte apoptosis is a major mechanism for initiation and development of OA. Proliferation and apoptosis of primary articular chondrocytes are measured with respective ELISA kits for BrdU incorporation and apoptosis. Chondrocyte maturation is evaluated after 24 hr of MV incubation (by qRT-PCR) for the expression levels of chondrocyte-specific genes, such as Col2α1, Col10α1, Col1α1, Acan, Prg4, and Sox9. Functional activity is determined by the formation of acidic proteoglycan-producing colonies (identified by alcian blue staining) after 21 days of MV incubation. The relative amount of released stain released from the colonies is measured by an established method as described in the legend of FIG. 1B. A dose-dependent effect of osteoclast-derived MV on the chondrogenic activities is also determined.

The second set of experiments is to determine if osteoclastic MV has suppressive action on synovial inflammation. The relative expression levels (by qRT-PCR) of proinflammatory cytokines (e.g., IL-1β, IL-6, TNFα, IL-2, IL-7, IL-15, and IL-21) and extracellular matrix proteases and inhibitors (e.g., MMP1, MMP3, MMP13, ADAMTS4, ADAMTS5, and TIMP) will be measured in synoviocytes isolated from injured joints and compare them with those of synoviocytes isolated from uninjured control joints, because inflamed synoviocytes secrete inflammatory cytokines and extracellular matrix proteases. The relative amount of the released proinflammatory cytokines and extracellular matrix proteases proteins in CM of each synoviocyte culture will be measured using respective commercial ELISA kits. The MV treatment may reduce expression and release of these inflammatory cytokines and extracellular matrix proteases. The dose-dependent effects of osteoclastic MV on the anti-inflammatory activity will also be determined. The third set of experiments is to ascertain that treatment of osteoprogenitors (isolated according to established procedure (Sheng, M. H., et al. (2004) Bone 35, 711-719)) with MV derived from CM of differentiated RAW-OC would promote their proliferation, cell viability, differentiation, and functional activity. Osteoblastic proliferation and apoptosis are assayed with respective commercial ELISA kits for BrdU incorporation and cell apoptosis after 24 hrs of MV incubation. Osteoblast differentiation is monitored after 24-48 hr MV incubation by determining the cellular expression of alkaline phosphatase (ALP) activity (a marker of differentiated osteoblasts) and the expression levels of osteoblastic genes by qRT-PCR, such as Col1α1, Ocn, Opn, Osx, and Bsp. Osteoblastic activity is assessed with the mineralized nodule formation assay after 21 days incubation under mineralization condition. Dose-dependent curve for the osteogenic activity is also determined.

The dose-dependent curves for the pro-chondrogenic activity, the anti-inflammatory activity, and the osteogenic activity will be compared. If the three dose-dependent curves are very different, the results will be interpreted to mean that different factors within the MV are responsible for these activities. If similar dose-dependent curves are obtained, it will be determined that the same factor within the MV is responsible for these three biological activities.

Experimental strategy 1b will be carried out determine if the pro-chondrogenic, osteogenic, and anti-inflammatory activities are specific to MV and not to other EV subfractions of osteoclasts. These experiments are important because the biologically active components in MV, AB, or Exo and their functions are likely to be different, as their cellular release mechanisms are triggered by different and distinct cellular events (Heijnen, H. F., et al. (1999) Blood 94, 3791-3799). Therefore, it is important to know whether the soluble pro-chondrogenic, osteogenic, and/or anti-inflammatory activities released by osteoclasts reside on the same EV subfraction or widely distributed in the EV subfractions. This information could disclose valuable regulatory and/or functional insights into the nature and/or identity of the active components. For this work, AB, MV, and Exo subfractions will be prepared from the same pooled CM of RAW-OC by differential ultracentrifugation. The identity of each subfraction is confirmed with a known marker of AB (e.g., Tsp1), MV (e.g., Arf6), Exo (e.g., CD9), respectively. Next, experiments will be performed to characterize the pro-chondrogenic, osteogenic, and anti-inflammatory activities of MV (as described herein) and they will be compared with those of AB and Exo. Since the identity of the active component(s) is currently not known, the relative activity of each subfraction is normalized against its total protein content. It is expected that the majority of each foregoing biological activity is found to be associated with the MV subfraction.

Experimental strategy 1c will be carried out to determine whether the pro-chondrogenic, osteogenic, and anti-inflammatory activities of MV is specific to cells of monocyte/macrophage lineage, such as differentiated osteoclasts and osteoclast progenitor), and not cells of mesenchymal lineage, such as articular chondrocytes and synoviocytes. This work is important because there is evidence that infiltrated macrophages and osteoclasts during the initial phase of the joint injury, in addition to cleaning up damaged tissues, trigger an innate repair mechanism to repair damaged articular cartilage and subchondral bone through the osteoclastic release of MV that is similar to the coupled bone formation to bone resorption mechanism during bone turnover. Briefly, MV subfractions can be isolated from pooled CM of RANKL/mCSF-differentiated and undifferentiated marrow-derived primary osteoclast, articular chondrocytes, and synoviocytes by differential ultracentrifugation. The pro-chondrogenic, osteogenic, and anti-inflammatory activities of each MV subfraction are determined as described herein. These three activities may be restricted predominantly to the MV subfraction released by differentiated and undifferentiated osteoclasts, but not cells of mesenchymal lineage, such as articular chondrocytes and synoviocytes.

The extent and degree of RANKL-mediated RAW264.7 cell differentiation varies significantly from one preparation to another. Thus, it is expected to have substantial differences in the relative quantity and quality of MV released into CM from various preparations. Consequently, it is important to control the quality and quantity of each preparation of MV. Accordingly, CM will be pooled from sufficient numbers of osteoclast cultures for the isolation of MV, such that the same MV preparation is used in each study for proper comparison. Each EV subfraction isolated by differential ultracentrifugation is relatively crude and not homogeneous. Thus, there could be some degrees of overlapping activities seen in some of the EV subfractions.

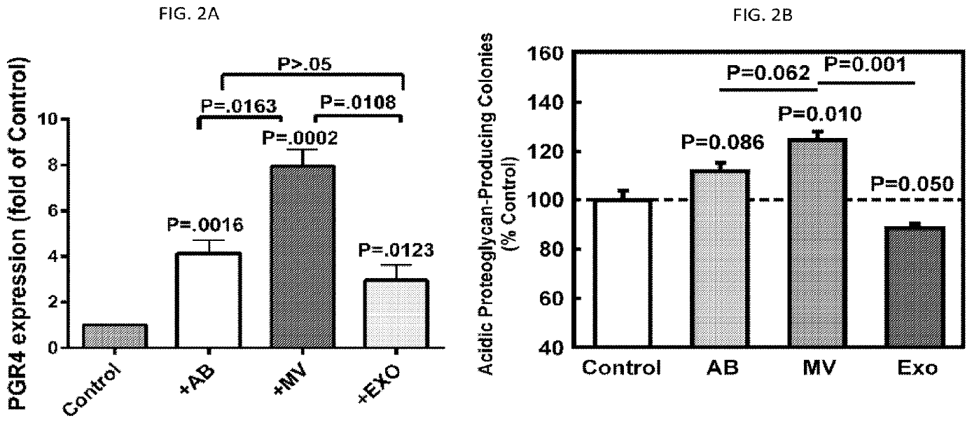

The results described herein demonstrate that osteoclastic microvesicles treatment alleviated OA development after an intraarticular tibial plateau injury. Also, the data provide evidence that the apparent osteoanabolic, pro-chondrogenic, anti-inflammatory effects are specific for osteoclastic MV and not for osteoclastic apoptotic bodies or exosomes. For example see FIGS. 2, 3, 4, and 6).

FIGS. 2A-B show that treatment of primary articular chondrocytes with osteoclastic MV, AB, or Exo upregulated cellular expression of Pgr4 and increased the ability of formation of acidic proteoglycan-producing colonies in primary articular chondrocytes. FIG. 2A shows that while the test extracellular vesicles upregulated Pgr4 expression, osteoclastic MV showed the greatest enhancing activity. Cellular Pgr4 expression level was determined by RT-PCR (n=4). Control cells were treated with same volume of PBS. Results are shown as mean±SEM (n=4). P values are determined by two-tailed Student's t-test compared to respective control group. FIG. 2B shows that treatment with primary chondrocytes with osteoclastic MV, but not osteoclastic AB or Exo (at the same protein concentration), for 21 days increased the ability of primary chondrocytes to form acidic proteoglycan-producing colonies in culture medium containing 10% fetal calf serum in medium containing 10% FCS. Control cells were treated with PBS. P values are determined by one-way ANOVA compared to respective control group. The dashed line is 100% of the controls.

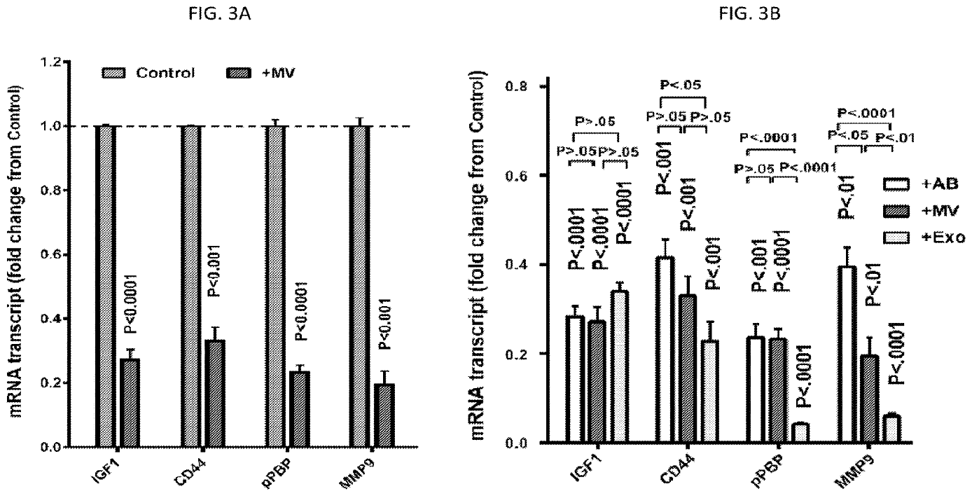

FIGS. 3A-B show that treatment of primary mouse synoviocytes with osteoclastic MV, AB, or Exo affects cellular expression levels of pro-inflammatory genes. FIG. 3A shows that osteoclastic MV downregulated the expression of several pro-inflammatory genes, e.g., Igf1, Cd44, pPBP, and Mmp9, in primary synoviocytes. Primary mouse synoviocytes were isolated from mouse knee joints (Zhao, J., et al. (2016) Biomed Rep 5, 171-175). Control cells were treated with PBS and experimental cells were treated with osteoclastic MV for 48 hours. Total RNA was extracted from control and experimental cells, and the expression levels of test genes were determined by RT-qPCR and normalized against eta-actin. Results are shown as mean±S.E.M., n=3. FIG. 3B shows that osteoclastic MV, AB, and Exo, each substantially suppressed the expression of genes associated with synovial inflammation in primary mouse synoviocytes. The relative cellular expression levels of synovial inflammation-associated genes, e.g., IGF1, CD44, pPBP, and Mmp9, in synoviocytes after incubation with the same protein concentration of MV, AB, or Exo (or PBS in control cells) for 48 hours. P values are determined by One-way ANOVA compared to respective control group. The dashed line is 100% of the controls. Results are shown as mean±S.E.M., n=3.

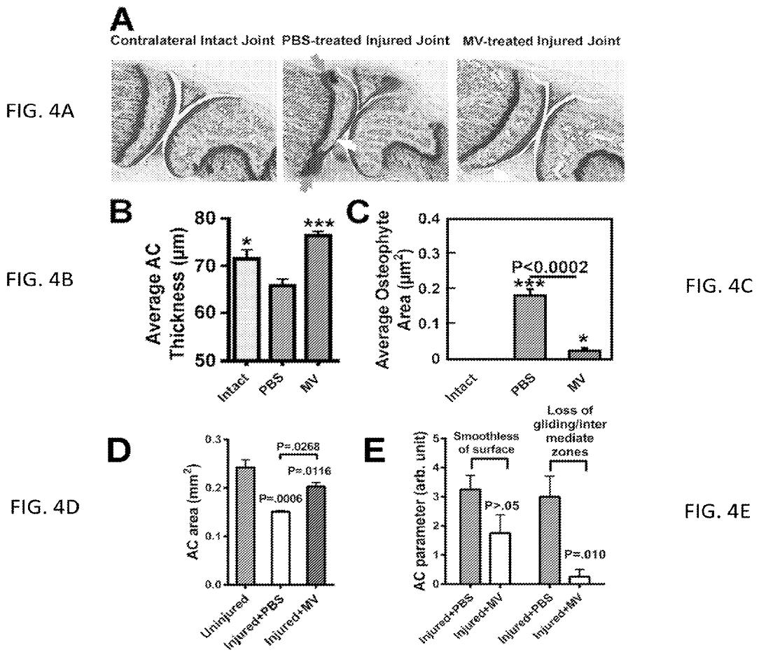

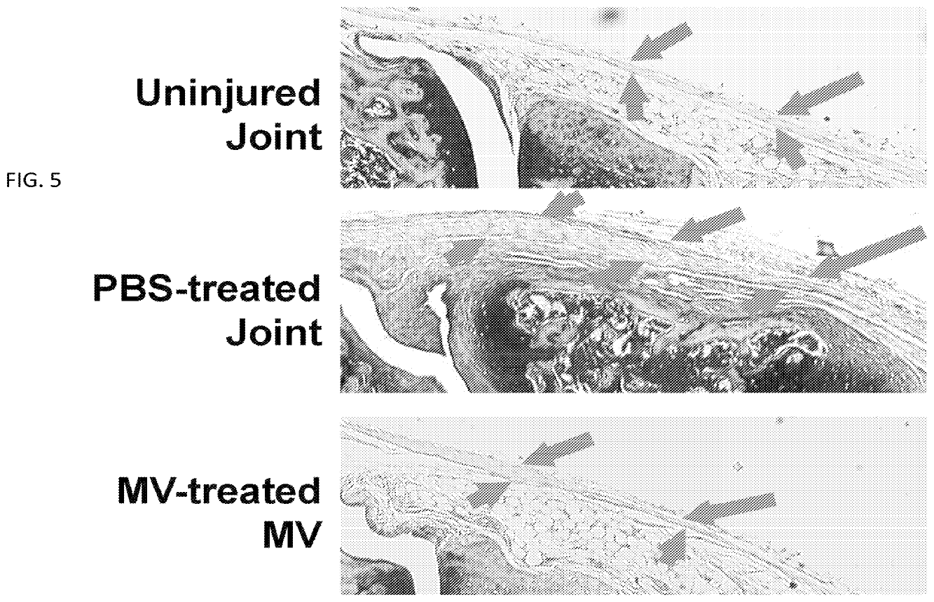

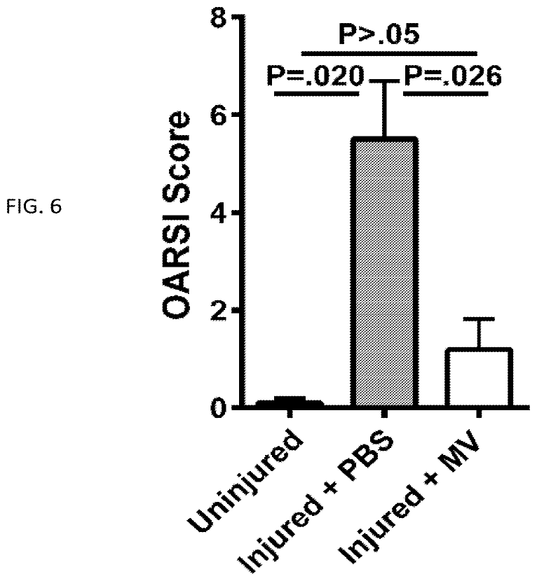

Biweekly intraarticular injections of osteoclastic MV prevented erosion and/or promoted regeneration of the damaged articular cartilage in osteoarthritis (OA). To test of feasibility of the osteoclast-derived MV-based strategy for OA, it was tested whether biweekly intraarticular injections of osteoclastic MV into injured tibial joints would prevent or delay OA progression and losses of articular cartilage at 5 weeks after an intraarticular tibial plateau injury. The experimental design was used to assess the efficacy of an EphA4 signaling-based therapy for OA/PTOA in mice (Stiffel, V. M., et al. (2022) J Bone Miner Res), with an exception that osteoclastic MV rather than EfnA4-fc was administered. For this work, a validated mouse intraarticular tibial plateau injury model was used as the animal model of OA/PTOA (Stiffel, V., et al. (2020) Calcif Tissue Int 106, 158-171). Briefly, a dose (~60 μg MV protein in 20 μl) of MV isolated from conditioned media (CM) of RAW264.7 cell-derived osteoclasts (RAW-OC) or PBS (control) was injected intraarticularly into the injured joint of groups of 4 adult male C57BL/6 mice at one day after the injury and every two weeks thereafter. The relative severity of OA on injured joints was assessed at 5 weeks post-injury (Stiffel, V. M., et al. (2022) J Bone Miner Res; and Stiffel, V., et al. (2020) Calcif Tissue Int 106, 158-171). The MV-treated injured joints had greater average articular cartilage thickness (white arrows in FIG. 4A) than PBS-injected control joints (FIG. 4B). They also formed less osteophyte-like tissues (red arrows in FIG. 4A) compared to PBS-injected injured joints (FIG. 4C). FIG. 4D shows the average articular cartilage area on the injured tibial plateau site, measured on multiple sections of each mouse. FIG. 4E shows that osteoclastic MV treatment reduced the loss of articular cartilage surface smoothness and loss of gliding/intermediate zones induced by OA. Synovial hyperplasia is a pathologic hallmark of OA/PTOA. FIG. 5 confirms that the tibial plateau injury increased the apparent thickness of synovium at the injured joint (indicated by red arrows); an index of synovial hyperplasia in response to inflammation. The MV treatment reversed the synovial hyperplasia in the injured joint. FIG. 6 shows that biweekly intraarticular injections of osteoclastic MV reduced the severity of OA developed after the closed intraarticular tibial compression loading-induced injury at 5 weeks post-injury. Because synovial hyperplasia is caused largely by the actions of pro-inflammatory cytokines, the data demonstrate that the osteoclast-derived MV also has anti-inflammatory actions in OA/PTOA. However, OA has a complex pathophysiology. A comprehensive therapy should be able to concertedly treat the three major etiologies of OA/PTOA, e.g., i) chronic synovial inflammation, ii) inability of damaged articular cartilage to regenerate itself, and iii) dysfunctional remodeling of subchondral bone structures. Because the findings disclosed herein show that osteoclast-derived MV has pro-chondrogenic, anti-inflammatory, and osteoanabolic properties in cells and tissues of the joint relevant to OA, the diverse skeletal effects of osteoclastic MV together can provide comprehensive protective effects on OA/PTOA.

The next steps include: 1) to establish and characterize the diverse biological effects of osteoclastic MV on relevant skeletal cells (articular chondrocytes, subchondral osteoblasts, and synoviocytes); and 2) to evaluate the osteoclastic MV-based strategy for OA/PTOA.

Data and power analyses. The sample size for various endpoints is determined by power analysis based on standard deviation estimates from similar studies. The group size of “n”=8 animals per group will detect a difference of 30% with a power of 0.80 in various histological parameters. For histology measurements, a 10% and 5% difference, respectively, should be detected with “n”=8 animals at p<0.05 with a power of 0.94. For in vitro assays, “n”=6 should allow detection of difference of 10% at p<0.05 with a power of 0.90.

Normal distribution and homogeneity of variance are first confirmed by the Levene's test. Data with non-normal distribution are analyzed by the Kruskal-Wallis test followed by Mann-Whitney U-test. For data sets showing normal distribution, comparison of differences between two variables is done with the two-tailed, two-sample with equal variances independent t-test. Comparison among multiple groups is done with analysis of variance (ANOVA) with Tukey's HSD post-hoc test. The differences are significant when p<0.05.

Osteoclast-derived MV preparations. Initially, the osteoclast-derived AB, MV, and Exo subfractions are isolated from the pooled CM of RAW-OC by differential ultracentrifugation, e.g., AB-800×g for 20 min pellet, MV-16,000×g for 20 min pellet, and Exo-100,000×g for 60 min pellet (Thery, C., et al. (2006) Current protocols in cell biology Chapter 3, Unit 3 22). Results are confirmed with AB, MV, and Exo isolated from primary marrow-derived osteoclasts.

Mouse model of OA/PTOA. The detailed methodology and characteristics of the validated non-invasive mouse intraarticular tibial plateau injury model of OA/PTOA are described in (Stiffel, V., et al. (2020) Calcif Tissue Int 106, 158-171). Briefly, the intraarticular tibial plateau injury is produced using an Instron servohydraulic mechanical tester with the mouse under isoflurane inhalation anesthesia. The right knee of a mouse is positioned on an apparatus attached to the load cell of mechanical tester. To injure tissues at the tibial plateau, the excursion of a blunt indenter blade is set on the top of the tibial plateau. An impact force is applied under a defined force of 55N at a speed of 60 N/s to the tibial plateau as the leg of the animal is positioned in a cradle support. This rapid impact force yields substantial loading energy sufficient to create significant injuries to the synovium, meniscus, and articular cartilage but not enough to create fractures at the tibial plateau.

### Example 2: Intraarticular Injections of Osteoclast-Derived MV into Injured Joints Prevent OA/PTOA Development and Maintain the Structural Integrity of Articular Cartilage and Subchondral Bone after a Tibial Plateau Injury

To test whether the progression of OA/PTOA after an intraarticular tibial plateau injury can be slowed down, the relative effectiveness of a single intraarticular injection will be compared with that of biweekly intraarticular injections of osteoclast-derived MV. A dose range of osteoclast-derived MV will be determined. Also, it will be determined whether the treatment initiated after OA that has already been established is still effective in reversing the symptoms of OA/PTOA

A mouse model of closed intraarticular tibial plateau injury model of OA/PTOA will be used to test the osteoclast-derived MV-based strategy.

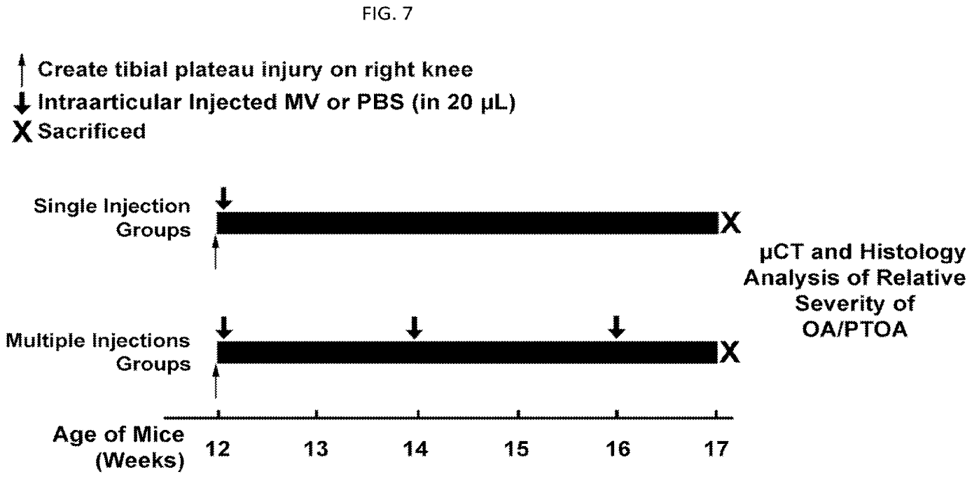

Experimental strategies. The experimental strategy focuses on three aspects of the therapeutic strategies. In experimental strategy 2α, the relative efficacy of a single intraarticular injection will be compared with that of multiple biweekly intraarticular injections of osteoclast-derived MV in retarding the progression of OA/PTOA after an intraarticular tibial plateau injury. The biweekly injection regimen for testing was chosen because data generated shows that the biweekly intraarticular regimen is effective. The validated non-invasive intraarticular tibial plateau injury model (Stiffel, V., et al. (2020) Calcif Tissue Int 106, 158-171) will be used. The experimental design is schematically shown in FIG. 7. Briefly, an intraarticular tibial plateau injury is created on the right joint of 12 weeks old C57BL/6J mouse. The uninjured left joint is used as the paired internal control for each injured right knee. For the single injection group, an effective dose (60 μg MV protein in 20 μl) of osteoclast-derived MV or 20 μl PBS (untreated control) is injected intraarticularly into the injured joint of 8 mice per group on one day after the injury. For the multiple injections group, the injection is injected on day 1, week 2 and week 4 post-injury. The mice are euthanized at 5 weeks post-injury. Both the injured right joint and uninjured left joint of each mouse, including distal femur and proximal tibia are removed and fixed with 10% cold neutral buffered formalin for 4-5 days. The fixed joints are then analyzed by μ-CT and histology (Stiffel, V. M., et al. (2022) J Bone Miner Res; and Stiffel, V., et al. (2020) Calcif Tissue Int 106, 158-171). Contralateral uninjured left joints were used as corresponding internal controls because the injury on the right joint has no effects on the uninjured left joint (Stiffel, V. M., et al. (2022) J Bone Miner Res; and Stiffel, V., et al. (2020) Calcif Tissue Int 106, 158-171). The relative severity of OA/PTOA is determined by μ-CT, histology, a modified histology-based Mankin OA disease scoring system, and serum levels of OA biomarkers, COMP and CTX-II. These methods are described in detail (Stiffel, V. M., et al. (2022) J Bone Miner Res; and Stiffel, V., et al. (2020) Calcif Tissue Int 106, 158-171). This experiment will inform on whether a single application of MV is sufficient for the efficacy or multiple injections are needed.

Experimental strategy 2b will be carried out to determine an optimal dose range of osteoclast-derived MV. The experimental design of this set of experiment will be similar to that of the multiple injection protocol of Experimental 2α, except that the injured joint is injected with the various increasing test doses of MV. The initial test dosage range, based on the protein content of MV in 20 μl, are 0 (PBS), 30 μg, 60 μg, 90 μg, and 120 μg. Additional doses are included if necessary.

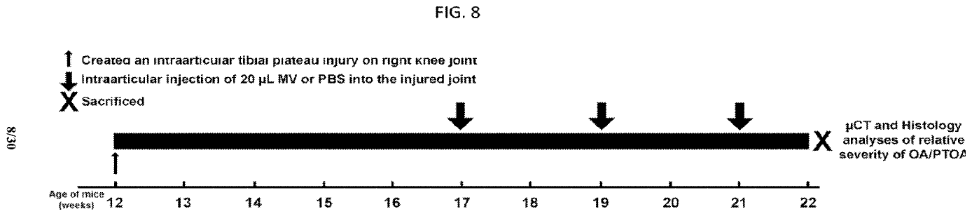

Experimental strategy 2c will be carried out to determine whether the treatment initiated after OA that has already been established is capable of reversing the symptoms of the established OA. This is important, because human patients usually seek medical attention after the disease has progressed to a stage that caused substantial pain and symptoms. The experimental strategy is shown schematically in FIG. 8. Briefly, an intraarticular tibial plateau injury is created on the right knee joint of 12-week-old C57BL/6 mice (8 mice per group). OA is allowed to develop for 5 weeks. At 5 weeks post-injury, osteoclast-derived MV protein (60 μg in 20 μL) or PBS (20 μL) is injected intraarticularly every other week. Mice are euthanized and analyzed 5 weeks later by μ-CT and histology for relative severity of OA as described herein. The treatment regimen determined can be used reduce severity of OA/PTOA and can promote regeneration of the damaged articular cartilage.

The active component of MV is likely a membrane receptor or membrane-associated ligand of a membrane receptor. Membrane receptor-mediated signals can be subject to desensitization due to receptor internalization. This should not be a significant issue because the duration between injections is two weeks apart which would minimize the risk of receptor desensitization. The duration between injections can be increased. This experimental strategy focuses on an early phase of OA development, as the MV is administrated at one day post-fracture. The time course of the biological effect of the therapy will be determined at the early phase of treatment and at a later phase.

Data and power analyses. The sample size for various endpoints is determined by power analysis based on standard deviation estimates from similar studies. The group size of “n”=8 animals per group will detect a difference of 30% with a power of 0.80 in various histological parameters. For histology measurements, a 10% and 5% difference, respectively, should be detected with “n”=8 animals at p<0.05 with a power of 0.94. For in vitro assays, “n”=6 should allow detection of difference of 10% at p<0.05 with a power of 0.90.

Normal distribution and homogeneity of variance are first confirmed by the Levene's test. Data with non-normal distribution are analyzed by the Kruskal-Wallis test followed by Mann-Whitney U-test. For data sets showing normal distribution, comparison of differences between two variables is done with the two-tailed, two-sample with equal variances independent t-test. Comparison among multiple groups is done with analysis of variance (ANOVA) with Tukey's HSD post-hoc test. The differences are significant when p<0.05.

Osteoclast-derived MV preparations. Initially, the osteoclast-derived AB, MV, and Exo subfractions are isolated from the pooled CM of RAW-OC by differential ultracentrifugation, e.g., AB-800×g for 20 min pellet, MV-16,000×g for 20 min pellet, and Exo-100,000×g for 60 min pellet (Thery, C., et al. (2006) Current protocols in cell biology Chapter 3, Unit 3 22). Results are confirmed with AB, MV, and Exo isolated from primary marrow-derived osteoclasts.

Mouse model of OA/PTOA. The detailed methodology and characteristics of the validated non-invasive mouse intraarticular tibial plateau injury model of OA/PTOA are described in (Stiffel, V., et al. (2020) Calcif Tissue Int 106, 158-171). Briefly, the intraarticular tibial plateau injury is produced using an Instron servohydraulic mechanical tester with the mouse under isoflurane inhalation anesthesia. The right knee of a mouse is positioned on an apparatus attached to the load cell of mechanical tester. To injure tissues at the tibial plateau, the excursion of a blunt indenter blade is set on the top of the tibial plateau. An impact force is applied under a defined force of 55N at a speed of 60 N/s to the tibial plateau as the leg of the animal is positioned in a cradle support. This rapid impact force yields substantial loading energy sufficient to create significant injuries to the synovium, meniscus, and articular cartilage but not enough to create fractures at the tibial plateau.

### Example 3: Functionalized Artificial Efnb2-Liposome-Based Therapeutic Strategy to Promote Fracture Repair

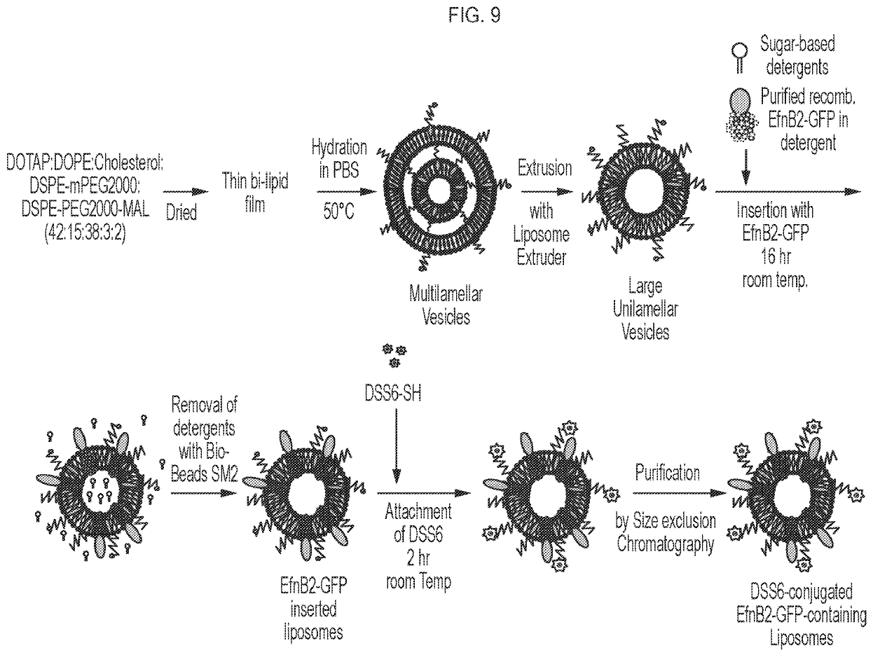

An EfnB2-MV (EfnB2 containing MV)-based therapeutic strategy to promote local bone formation, repair and regeneration was tested. An important advantage of this MV-based strategy is the high potency of the EfnB2-MV approach over recombinant EfnB2-fc approach; the results show that the in vivo local bone formation activity of the EfnB2-MV injection strategy is >3 orders in magnitude more potent than that of strategy using recombinant EfnB2-fc chimeric protein. To assess the EfnB2-MV approach, a functionalized liposome-based nanovesicle will be developed to deliver a PDGF-DSS6 fusion protein to bone surface to promote bone formation. The experimental strategy for producing EfnB2-containing functionalized liposome-based microvesicles is shown in FIG. 9. The in vitro and in vivo osteoanabolic activity of EfnB2-liposome will be characterized and compared to purified EfnB2-MV. A mouse model of tibial multiple fractures will be used to test the feasibility of EfnB2-MV-based strategy for local bone repair.

The experimental approach is divided into 3 phases. In the first phase, an EfnB2-containing liposome-based artificial MV (EfnB2-liposome) will be developed. The experimental approach is schematically shown in FIG. 9. This approach takes advantage of two important discoveries. First, it is feasible to reproducibly produce cationic liposomes with attachment of a bone-targeting peptide comprising six repeats of amino acids aspartate, serine, serine (DSS6). DSS6 has high binding affinity to hydroxyapaptide crystals and has been used as a bone targeting tool. Thus, an attachment of DSS6 peptide to the liposome would have an important advantage as it would enhance the targeting ability of the liposome to bone surfaces where bone formation takes place. Second, sugar-based detergents (e.g., n-dodecyl-β-D-thiomaltopyranoside (DOTM)) can promote the unidirectional insertion of transmembrane proteins (in the correct orientation) into liposomes. Briefly, 1,2-dioleoyl-3-trimethylammonium-propane (DOTAP), Dioleoylphosphatidylethanolamine (DOPE), cholesterol, DSPE-mPEG2000 and DSPE-PEG2000-MAL (at molar ratio of 42:15:38:3:2), in chloroform, are mixed, lyophilized, and rehydrated with 10 mM PBS pre-incubated in water bath at 50° C. to form thin films of multilamellar vesicles (MLV). The MLV is then extruded in a LipoFast mini extruder (Alves, N. J., et al. (2013) Analyst 138, 4746-4751) to form large unilamellar vesicles (LUV). For the insertion of recombinant EfnB2-GFP, a drop of LUV in 75 μM DOTM is mixed with 20 μl of concentrated buffer (200 mM, K₂SO₄, 20 mM PIPES (pH7.5), and 75 μM DOTM). [Green fluorescent protein (GFP) is fused to the C-terminus of the EfnB2 recombinant fusion protein to aid its identification and quantitation]. The DOTM-solubilized recombinant EfnB2-GFP fusion protein is added at 0.5-1 μM and incubated for 16 hr at 20° C. Recombinant EfnB2-GFP fusion protein is produced in HEK293 cells (for proper glycosylation) by standard cloning procedure. (The molecular size of EfnB2 of HEK293 cell extract was the same as that of mouse osteoclasts, suggesting that similar glycosylation occurs on EfnB2 in these two cell types). A his6-tag is added to its N-terminus to facilitate purification. Detergent is removed by incubating the solutions for 2 h with Bio-Beads SM2 (Rigaud, J. L., et al. (1995) Biochim Biophys Acta 1231, 223-246). To attach DSS6 to the liposome, DSS6 containing an N-terminal acetylcysteine and liposomes (in 3:1 ratio) is incubated for 2 h at 20° C. and purified by Sepharose CL-4B size exclusion chromatography to remove the un-conjugated DSS6. The amounts of EfnB2-GFP fusion protein and phospholipids are determined with respective ELISA and fluorometric assay. Aliquots of liposome suspensions are mixed with mannitol (molar ratio of mannitol-to-lipid 5:1) and lyophilized for 48 h for storage at −80° C. until use.

### Example 4: Osteoclasts Release Soluble Microvesicles (MV) that have Osteogenic Activity

The healing of about 10% of the several million traumatic or nontraumatic bone fractures occur annually in the U.S. is delayed or impaired. This is a significant health issue causing considerable healthcare dollars and human suffering. An effective therapy that promotes fracture healing would yield substantial economic and humanitarian benefits). Fracture healing is a complex, dynamic cellular process involving various bone cell types, with osteoclasts and osteoblasts playing indispensable roles. The core of fracture healing is the callus remodeling process that is initiated with the resorption of the cartilaginous callus followed by the spatially- and temporally-linked coupled local formation of a bony callus bridging the fracture gap, which in turn is remodeled to cortical bone with fully restored mechanical and microarchitectural properties. Osteoclastic resorption is an important process for callus remodeling and subsequent local bone regeneration during fracture healing. Accordingly, enhancing osteoclast formation or activity during fracture healing accelerates bony bridging of the fracture gap, whereas deficient osteoclast formation or activity impairs the formation of bony calluses. Thus, the mechanism of osteoclastic regulation of local bone regeneration was examined.

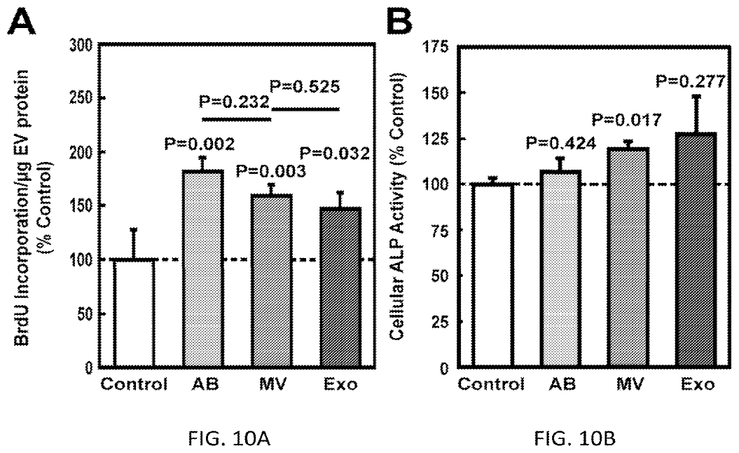

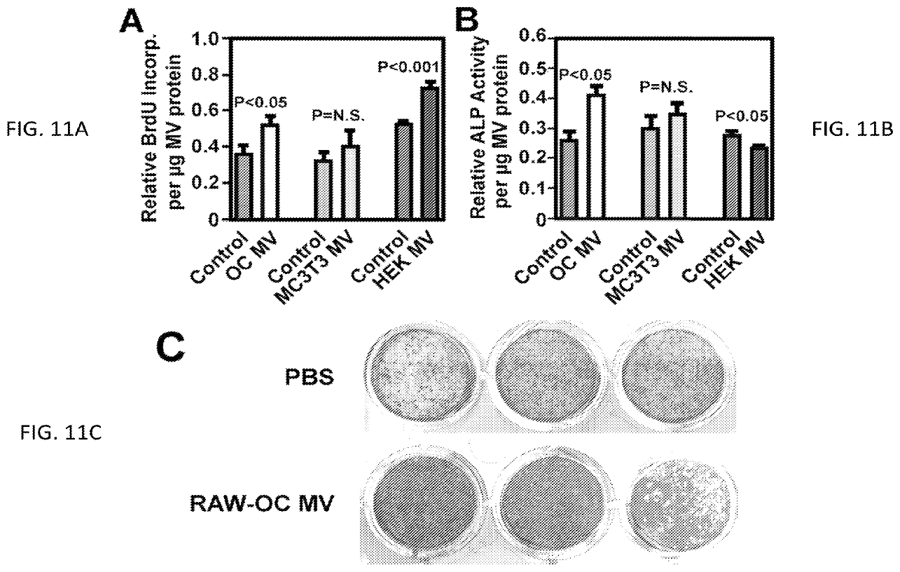

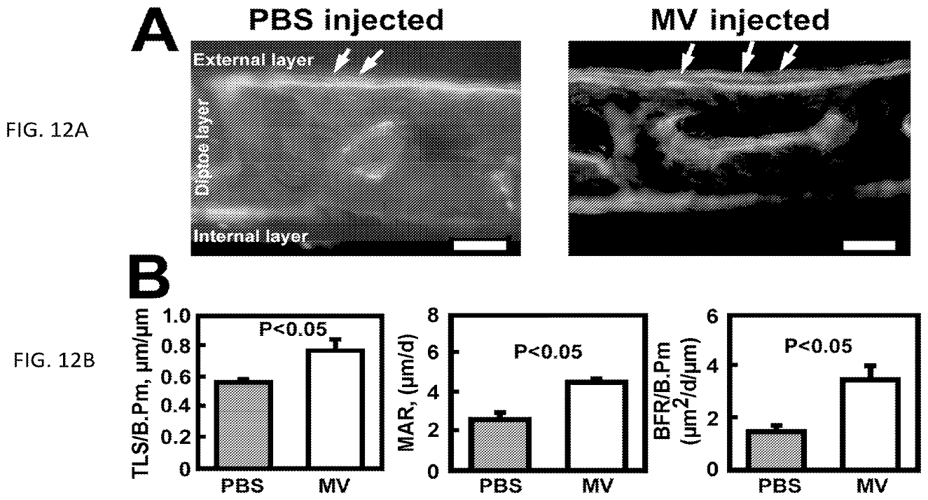

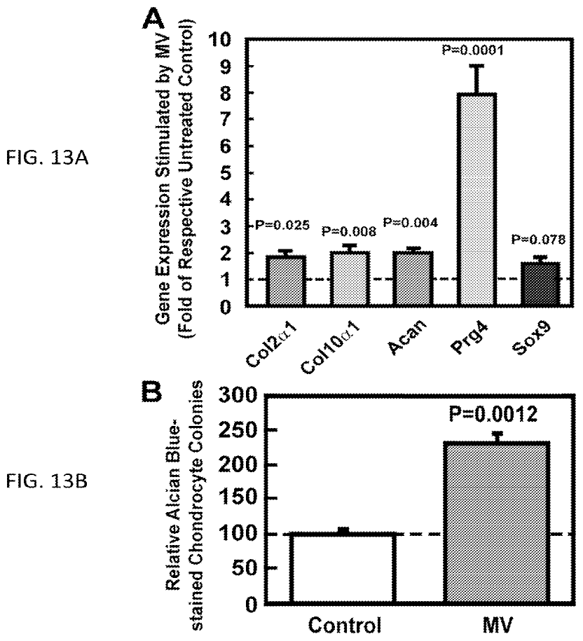

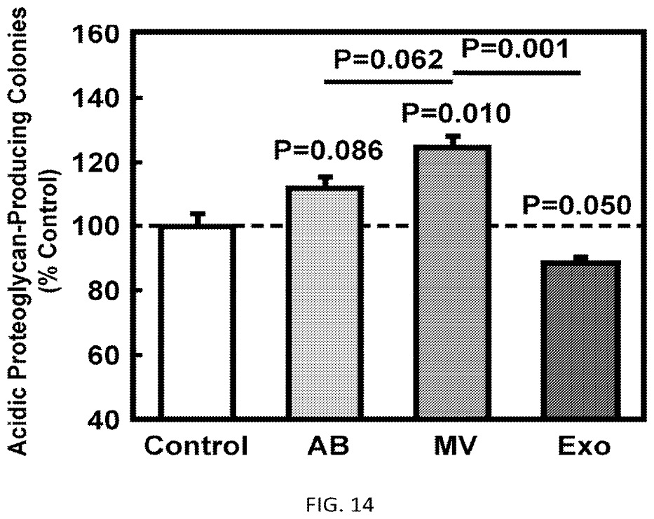

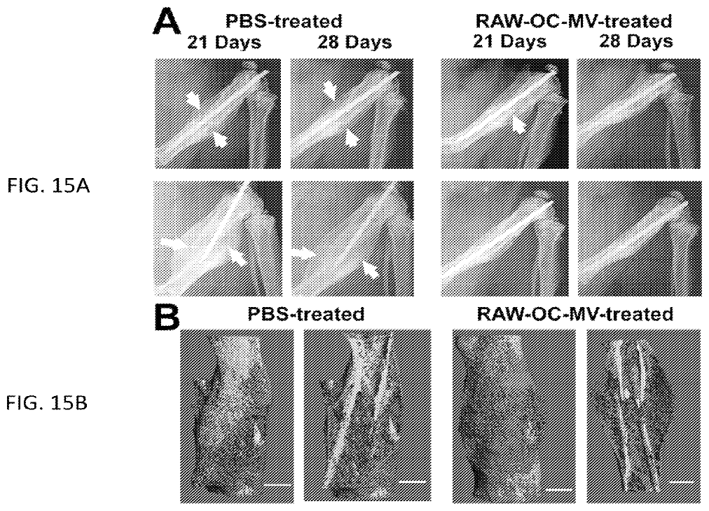

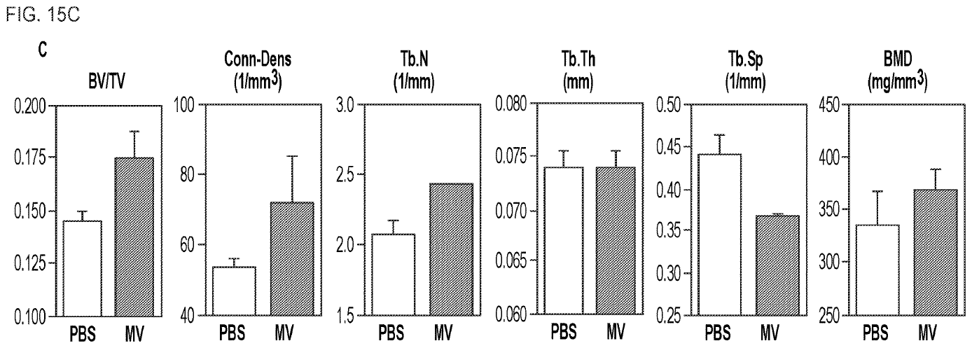

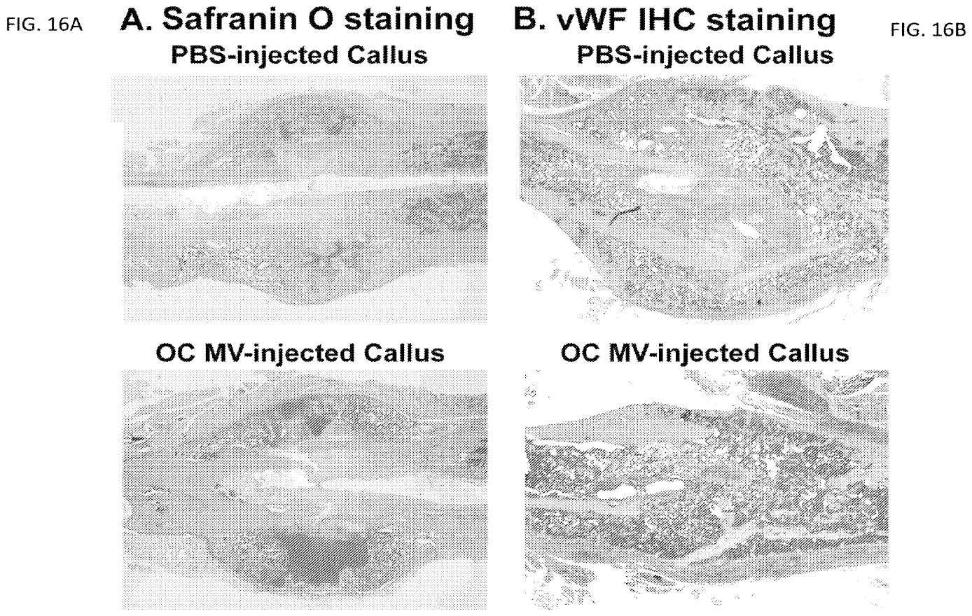

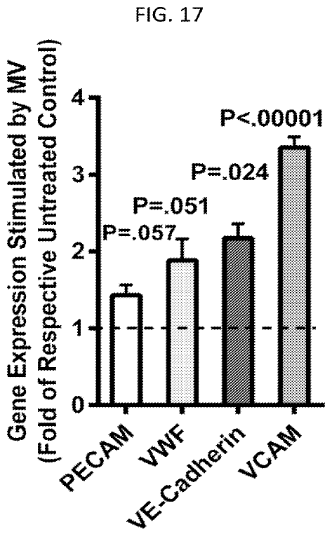

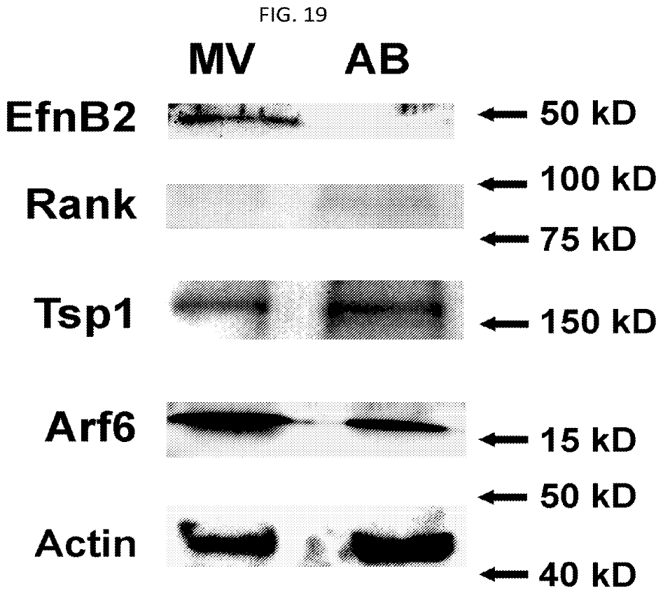

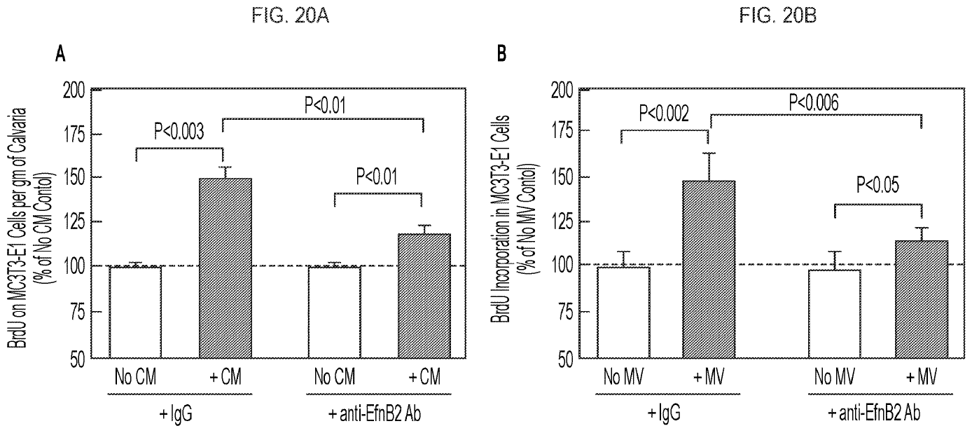

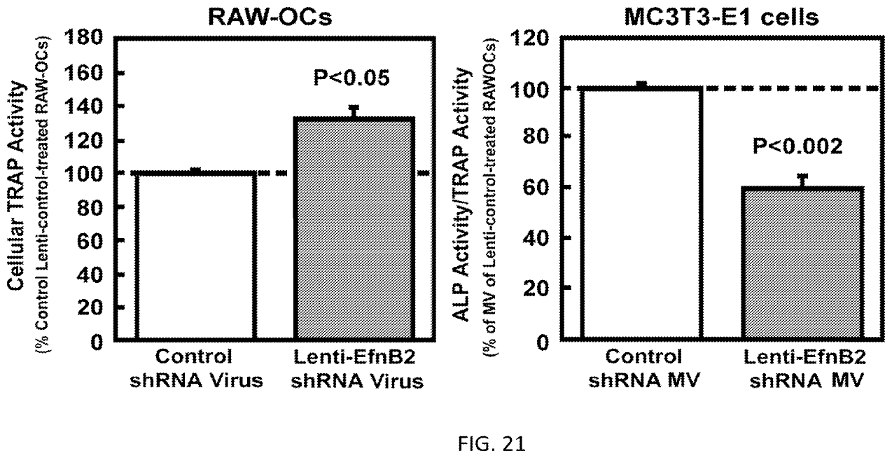

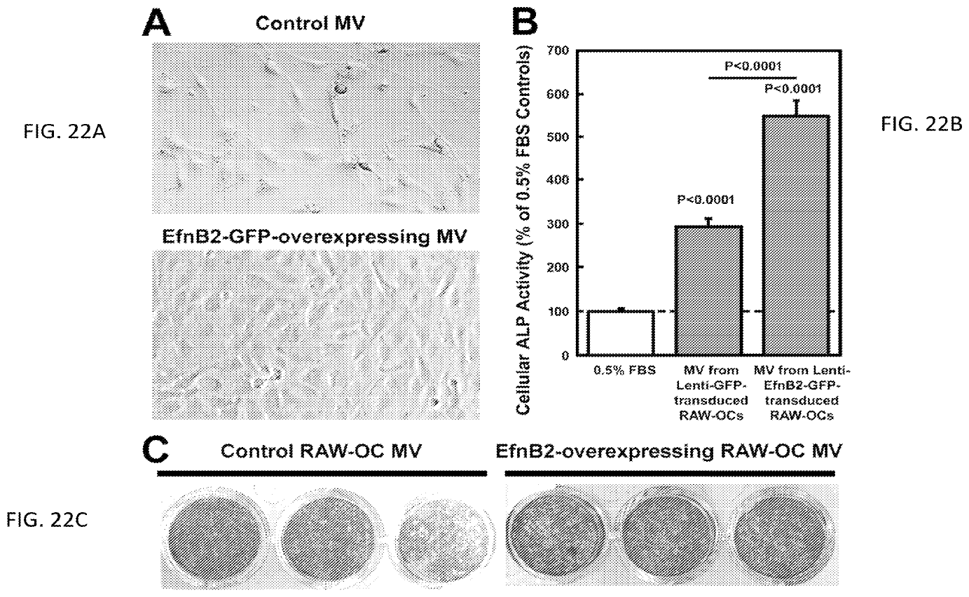

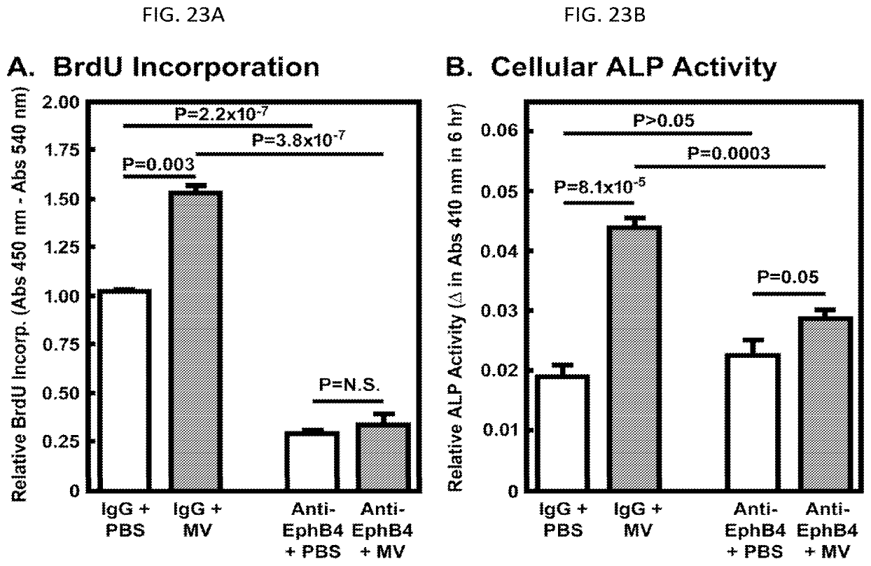

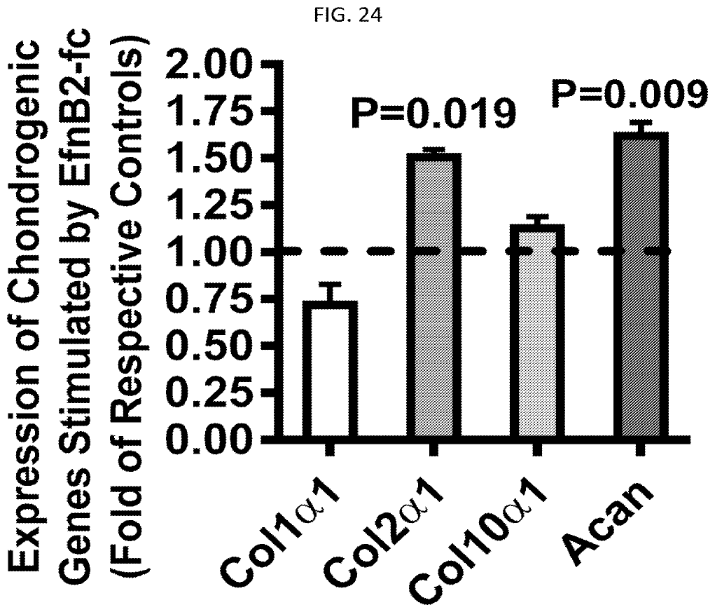

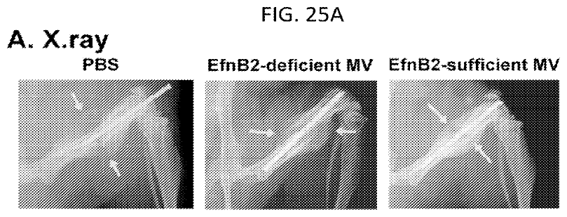

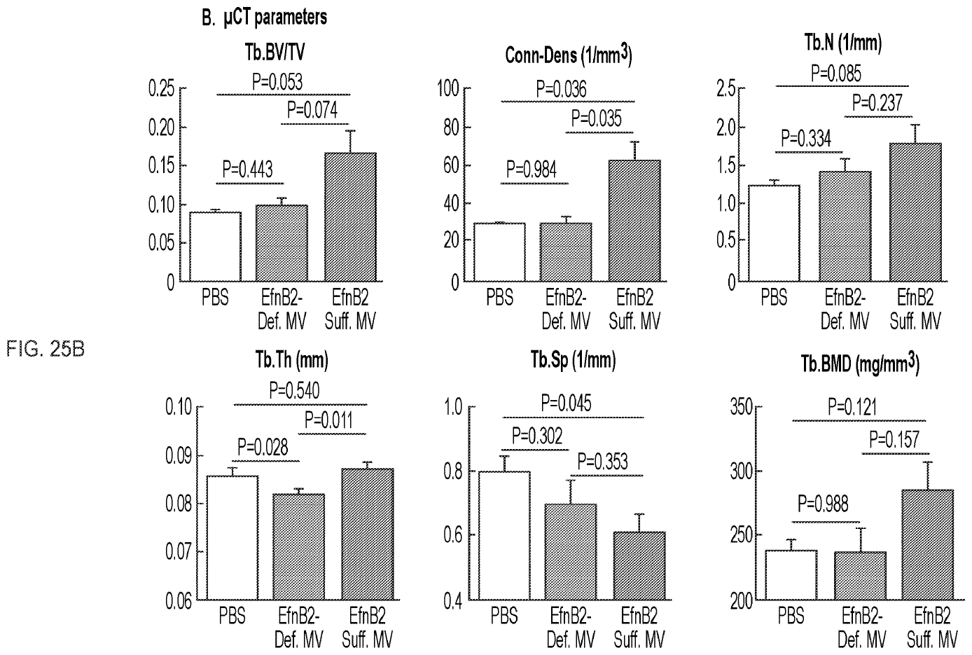

Osteoclasts regulate local bone regeneration in part via their release of soluble osteoanabolic factors. As described herein osteoclasts release soluble microvesicles (MV) that have osteogenic activity in osteoprogenitors (FIGS. 10A-B, and FIGS. 11A-C), pro-chondrogenic activity in chondrocytes (FIGS. 13 and 14), and pro-angiogenic activity in endothelial cells (FIG. 16B and FIG. 17). Direct injections of osteoclast-derived MV at the calvaria stimulated local bone formation in vivo (FIG. 12). Injections of osteoclast-derived MV into fractures promoted neo-angiogenesis in fracture callus, enhanced fracture healing, and restored torsional bone strength (FIGS. 15, 16, and 18). Cartilage and bone formation are two important processes of endochondral ossification for fracture healing. Thus, it was tested whether local injections of osteoclast-derived MV at fracture site accelerates fracture healing by promoting formation, remodeling, and neo-vascularization of cartilaginous callus during early phases and formation and remodeling of bony callus at later phases of fracture healing. Female sex has been identified as a major risk factor for compromised bone healing of elderly women in a study of a cohort of 1,133 patients. Studies in 12-months-old Sprague-Dawley rats and 8 to 12-weeks-old C57BL/6 mice also show that basal fracture healing in females is less robust than that in males. Despite the gender-dependent difference in the basal fracture healing capability, it was tested whether local injections of osteoclast-derived MV is similarly effective in female as in male mice. It is because similar osteoanabolic responses to osteoclast-derived MV were seen in cells derived from either sex in vitro. The active components on MV are likely membrane-associated growth/differentiation factors or ligands of their receptors. While it is possible that the osteogenic, chondrogenic, and angiogenic activities of MV are mediated by multiple distinct osteoanabolic components of MV, it was also tested whether EfnB2 is in part responsible for the observed osteoanabolic actions of osteoclast-derived MV. This concept is supported by findings showing that 1) osteoclast-derived MV contains substantial amounts of EfnB2 (FIG. 19), 2) pretreatment of osteoclast-derived MV with anti-EfnB2 antibody abrogated its osteogenic activity (FIG. 20), 3) EfnB2-deficient MV had reduced (FIG. 21), and EfnB2-overexpressing MV had greater (FIG. 22), osteoanabolic activity, 4) pretreatment of osteoprogenitors with anti-EphB4 antibody (cognate receptor of EfnB2) prevented osteoclastic MV to exert its osteogenic activity (FIG. 23), 5) soluble EfnB2 has pro-chondrogenic (FIG. 24) and pro-angiogenic activities, and 6) direct injection of EfnB2-sufficient, but not EfnB2-deficient, MV into fractures enhanced fracture healing (FIG. 25).

First, it will be determined, using a simple transverse femoral fracture model in young adult mice, a) an optimal injection regimen for osteoclast-derived MV by determining the duration-of-stay of the osteoclast-derived MV at fracture calluses, an optimal injection frequency, and an optimal dose range, b) if an optimized injection regimen of osteoclast-derived MV would increase mechanical strength of fractured bones and accelerate bony bridging of fracture gap similarly in female mice as in male mice, and c) cellular mechanism by which osteoclast-derived MV accelerates fracture healing in vivo by performing histological analysis of time-dependent effects of MV on osteoclasts and osteoblasts during formation and remodeling of cartilaginous and bony calluses.

Second, it will be determined whether: a) treatment of osteoblasts, chondrocytes, and endothelial cells with osteoclast-derived MV in vitro would upregulate their EphB4 forward signaling; b) MV released by EfnB2-deficient osteoclasts would have diminished chondrogenic and angiogenic activities of osteoclast-derived MV in vitro; c) MV released by EfnB2-overexpressing osteoclasts would increase osteogenic, chondrogenic, and angiogenic activities in vitro; d) osteoprogenitors, chondrocytes, and endothelial cells of EphB4-deficient mice would show reduced osteogenic, chondrogenic, and angiogenic responses to osteoclast-derived MV in vitro; and e) local injections of EfnB2-deficient osteoclast-derived MV would impede, and local injections of EfnB2-overexpressing osteoclast-derived MV would accelerate, fracture healing in vivo.

The experiments described herein will be carried out to determine a mechanism by which osteoclast-derived MV regulates local bone regeneration. The results will provide mechanistic information into how osteoclasts regulate local bone regeneration and can be used to develop osteoclast-derived MV-based therapeutic strategies for healing of fractures or local skeletal defects.

Cellular mechanism of fracture healing. Bone is one of the very few adult tissues that heals injuries (e.g., fractures) without formation of fibrous scars, such that fracture healing truly recapitulates bone development and can thus be considered as a form of true tissue regeneration. Despite the amazing regenerative capability, this repair process fails in ~10% of fractures, resulting in delay in healing or developing pseudo-arthrosis or non-unions. The healing of bone fractures is an intricate process involving a complex series of molecular events that coordinate the proliferation, differentiation, and functional activities of various diverse cell types. The fracture healing process can be divided into four phases: 1) hematoma phase, 2) fibrocartilaginous (soft) callus phase, 3) bony (hard) callus phase, and 4) callus remodeling phase. A hematoma is formed immediately after the fracture. Macrophages, monocytes, lymphocytes, and mesenchymal stem cells are rapidly recruited to the injury site, and the various types of proinflammatory cytokines are secreted to initiate an acute inflammatory response. Endochondral ossification then occurs in between the fracture ends and external to periosteal sites, forming the mechanically less stable fibrocartilaginous callus. Intramembranous ossification also takes place subperiosteally and directly adjacent to the distal and proximal ends of the fracture. Growth factors, including VEGFs, are secreted to induce neo-angiogenesis that leads to the mineralization and remodeling of cartilaginous callus, which progressively replaces fibrocartilaginous tissue with woven bone to form bony callus. The bony callus is eventually remodeled to regenerate cortical bone that is indistinguishable from native bone. The four phases are important for the success of fracture healing, but the core of fracture repair is the callus remodeling.

Callus remodeling. The remodeling of the fracture callus is accomplished through the collaborative and sequential actions of osteoclasts and osteoblasts: the osteoclastic resorption of soft callus is spatially and temporally coupled to an increased local bone formation to form hard callus, which is subsequently remodeled to regenerate intact cortical bone with the original architectural and mechanical properties. A defective local coupled bone formation is the frequent etiology of delayed or impaired fracture healing. Osteoclasts have an important regulatory role in the local coupled bone formation during fracture healing. Accordingly, enhancing osteoclast formation or activity accelerated cartilaginous callus remodeling as well as bony bridging of the fracture gap during fracture repair; whereas deficient osteoclast formation or activity impaired the remodeling of both soft and bony calluses and bony bridging of the fracture gap. Understanding the osteoclastic regulation of local coupled bone formation would therefore provide information into the pathogenesis of relevant bone disorders, including impaired fracture repair, and can lead to the development of therapies.

Osteoclast-derived soluble osteoanabolic factors. The osteoclastic regulation of local bone formation is complex and involves multiple mediators and mechanisms. There is evidence that this important process is mediated in part through locally produced soluble protein factors, which act on nearby osteoprogenitors to stimulate osteoblast formation. Osteoclasts are a major cell source for many of the local soluble osteoanabolic factors. Several osteoclast-derived osteoanabolic factors, such as TGFβ1, IGF-1, BMP6, LIF, SemA4D, CT-1, SIP/SPHK1, CTHRC1, Wnt10b, and complement factor 3a (C3α), CREG2, CST3, CCBE1, and DPP4, have been identified. Recent evidence indicate that osteoclasts may also regulate local bone formation via the release of soluble membrane-anchored factors, e. g., RANK (37-39), in some forms of extracellular vesicles (EV).

Osteoclasts release soluble EV to regulate local bone and cartilage formation. EV are nanosized, membrane-enclosed vesicles released by cells that transport DNA, RNA, or protein biologic cargoes between distant cells as a form of intercellular communication. EV are classified into 4 subtypes according to their biogenesis pathways: 1) exosomes (Exo, 40 nm-120 nm)—formed by fusion of multivesicular bodies and plasma membrane, in which multivesicular bodies release smaller Exo; 2) microvesicles (MV, 100 nm-1 μm)—formed by budding directly from plasma membrane; 3) apoptotic bodies (AB, 50 nm-2 μm)—released by apoptotic cells; and 4) oncosomes (1 μm-10 μm)—produced by malignant cells via membrane protrusion. Apoptotic osteoclasts released osteogenic AB that appears to have important mediating roles in the local coupled bone formation. As described herein, living osteoclasts, contrary to apoptotic osteoclasts, released large amounts of MV that has potent osteogenic activity on osteoprogenitors, pro-chondrogenic activity on chondrocytes, and pro-angiogenic activity on endothelial cells. Direct injections of osteoclast-derived MV at the external surface of calvarium stimulated local bone formation and direct injections of osteoclast-derived MV at fracture sites promoted angiogenesis in fracture callus and accelerate fracture healing. These findings demonstrate that MV secreted by osteoclastic cells can be developed as an osteoanabolic therapy for healing of fractures and/or treating large bony defects.

Significance. Bone fractures are common injuries. The annual cost for treatment and management of skeletal fractures in the U.S. alone exceeds $20 billion. Although fractures typically heal well following standard clinical care without interventions, ~10% of the 7.9 million fractures annually in the U.S. exhibit impaired healing that leads to nonunion or delayed union. To date, BMP-2 and BMP-7 are the only FDA-approved therapeutics for delayed unions and recalcitrant non-unions of long bone fractures. The BMPs have many key limitations. For example, delivery of BMPs requires surgical implantation at the site of fracture that could often result in complications, such as infection and heterotopic bone formation. Delivery of supra-physiological doses, which is required to achieve therapeutic effects, could lead to adverse side-effects, clinical complications, and prohibitive costs. The studies described herein can be used to develop an alternative bone-acting osteoanabolic therapy to accelerate healing of fractures. Since osteoclastic regulation of local coupled bone regeneration has paramount roles in fracture healing, understanding this molecular mechanism(s) can lead to the identification of therapeutic targets for more effective osteoanabolic therapies. The data described herein can provide a mechanism of osteoclastic regulation of local bone regeneration. It is known that osteoclasts can be generated by peripheral blood monocytes. Accordingly, the patient's own peripheral blood can be used to generate osteoclasts for the isolation of osteoclast-derived MV. The use of the patient's own blood cells as the source for therapeutic osteoclast-derived MV could minimize risk for severe immune responses to the therapy and related adverse effects. This is clinically beneficial with respect to safety profile of the therapy. Finally, EV-based strategies are emerging technologies for delivering therapeutics, genes, or vaccines. Thus, the data described herein can also be used for osteoclast-derived MV-based osteoanabolic therapeutics.

Relevance to VA patient care mission. Lower extremity fractures are a frequent secondary condition for patients with chronic spinal cord injuries (a significant health issue for veterans) with an incident rate of 2 to 3% per year. The risk for lower extremity fracture non-unions in this patient population is increased by several factors, such as older age, gender, duration of spinal cord injury, and the fracture site. Fractures are also serious complications for osteoporosis, which are common in aging VA patients. While most osteoporotic fractures occur in the spine and the hip, there is a high incidence of long bone fractures in this patient population, particularly wrist factures due to the increased risk of falls. Moreover, a significant number of lower extremity stress fractures (common in active service members of the military) heals poorly and requires prolonged treatment and may even lead to an early medical discharge from the military. Their continued care is transferred to the VA. In short, poor fracture healing is a serious health issue for the VA healthcare mission. The data described herein can reduce the morbidity associated with unhealed skeletal fractures and improve the quality of life of veterans with impaired fracture healing.

The experiments described herein have two conceptual elements. The first element is the concept that during resorption, osteoclasts release abundant amounts of osteoanabolic MV that has osteogenic, chondrogenic, and angiogenic activities that work concertedly to promote endochondral ossification. Accordingly, using a single therapeutic agent, such as osteoclast-derived MV, to simultaneously promote bone and cartilage formation and/or remodeling can be advantageous as both bone and cartilage formation are important processes for the endochondral repair of bone fractures. The second conceptual element is that the results can provide evidence that osteoclasts release soluble EfnB2 in the form of MV as an important osteoanabolic factor to act on nearby skeletal progenitors to promote local bone and cartilage formation as well as on endothelial progenitors to induce neo-angiogenesis. This concept is different from the generally accepted concept (Zhao, C., et al., (2006) Cell Metab 4, pp. 111-121) that the membrane-associated EfnB2 on the plasma membrane of osteoclasts acts strictly in a cell-cell contact-dependent way to interact with the EphB4 receptor of neighboring target cells to trigger the bidirectional signaling mechanism to suppress osteoclastogenesis and to promote osteoblastogenesis simultaneously.

Results. An impaired local coupled bone formation in Epha4 KO mice is caused in part by an inability of osteoclasts to release soluble osteoanabolic factors. Mutant mice with disruption of the Epha4 gene showed an increased resorption without the compensatory increase in local bone formation, indicative of an impaired coupled bone formation. It was found that the ability of CM of Epha4 KO calvaria upon PTH treatment (to stimulate bone remodeling) to stimulate bone cell proliferation (BrdU incorporation in MC3T3-E1 cells) was 75% less than that of CM of PTH-treated WT calvaria in an ex vivo calvarium organ culture assay and that the EfnA4-fc treatment in osteoclasts increased ability of its CM to stimulate BrdU incorporation and ALP activity in MC3T3-E1 cells. EfnA4-fc was chosen as the soluble EphA4 ligand because EphA4 binds EfnA4 with the highest affinity. These findings support the conclusions that bones of Epha4 KO mice release much less bone cell mitogenic activity during remodeling and that the EphA4 signaling may have a stimulatory role of in osteoclastic release of soluble bone cell mitogens. It was previously showed that the EphA4 forward signaling stimulated the RhoA/ROCK signaling in osteoclasts. Importantly, treatment of primary osteoclasts with 10 μM CCG-1423 (a specific inhibitor of RhoA pathway signaling) during final 3 days of the 6 days of mCSF/RANKL treatment blocked both basal and EfnA4-fc-mediated osteoclastic release of soluble osteogenic activities into CM, suggesting that the RhoA/ROCK signaling axis may have an important regulatory role in the EphA4-dependent osteoclastic release of soluble osteogenic factors. The RhoA/ROCK signaling is important for biogenesis and cellular release of MV, raising the possibility that the osteoclastic release of soluble osteogenic factors involves biogenesis and release of EV, particularly MV.

MV released by osteoclasts stimulates the proliferation, differentiation, and functional activities of osteoprogenitors in vitro. The osteogenic activity of the 3 major subtypes of EV, e.g., AB, MV, and Exo fractions, isolated from the same pooled CM of RAW-OC were compared. RAW cells were used because they are a commonly used osteoclast progenitor cell model, they are more homogeneous than primary cells, they are readily transduced by viral vectors, and their use avoids the need of sacrifice of mice for osteoclast preparations. The pooled CM was fractionated by differential ultracentrifugation into AB [800×g for 15 min pellet], MV [16,000×g for 20 min pellet], and Exo [100,000×g for 60 min pellet] fractions. For osteoblast proliferation, BrdU incorporation into osteoblasts isolated from bones of C57BL/6J mice was measured. For osteoblast differentiation, cellular ALP activity was assayed after 48 hr incubation and normalized against protein content of each EV fraction. Although the three EV fractions increased BrdU incorporation (FIG. 10A), MV significantly increased cellular ALP activity (FIG. 10B). Exo also increased cellular ALP activity, but the increase was not significant due to large variations. Because increased osteoblast proliferation without increased differentiation usually does not increase bone formation, it was concluded that MV, but not the other EV fractions, has osteogenic activity. MV isolated from pooled CM of primary osteoclasts also significantly increased BrdU incorporation (FIG. 11A) and ALP activity (FIG. 11B) of MT3T3-E1 cells. In contrast, MV of MC3T3-E1 cells did not increase BrdU incorporation or ALP activity. MV of HEK293 cells increased BrdU incorporation but reduced ALP in MC3T3-E1 cells, demonstrating that the osteogenic activity of MV may be unique to osteoclasts. Consistent with bone formation stimulatory activity, MV of RAW-OC enhanced the mineralized nodule forming activity of osteoblasts (FIG. 11C).

Intracranial injections of osteoclast-derived MV promoted local bone formation. The calvarium injection assay was used to determine if osteoclast-derived MV stimulates local bone formation in vivo. Daily injections at the surface of calvarium of groups of three 12 week-old male C57BL/6 mice for 5 days with osteoclastic MV (~60 μg MV protein in 20 μL) increased histomorphometric bone formation parameters (TLS/B.Pm, MAR, and BFR/B.Pm) at external layer compared to PBS-injected calvaria (FIG. 12), indicating that osteoclast-derived MV is a potent local osteoanabolic agent in vivo.

MV released by osteoclastic cells has pro-chondrogenic activity in chondrocytes. Primary chondrocytes were isolated from articular cartilage layers of C57BL/6J mice according to and cultured in DMEM/F-12 containing 2% FCS with no more than 3 passages. These cells expressed Col2α1 mRNA 554,000-fold more than Col1α1 mRNA, and 94,600-fold more than Col10α1 mRNA, confirming that these were bona fide articular chondrocytes. Treatment of these chondrocytes with osteoclast-derived MV (45 μg MV protein/mL) increased mRNA levels for chondrogenic genes (Acan, Prg4, Col2α1, Col10α1, Sox9) (FIG. 13A) and induced acidic proteoglycan-producing colony formation (FIG. 13B), indicating that the osteoclast-derived MV fraction has pro-chondrogenic activity.

The pro-chondrogenic activity was compared among MV, AB, or Exo fractions isolated from the same pooled CM of RAW-OC. At the same protein concentration, MV significantly enhanced the formation of acidic proteoglycan-producing colonies after 21 days in chondrogenic cultures (FIG. 14). AB slightly (but not significantly) increased; whereas Exo did not enhance but also significantly reduced, the ability of chondrocytes to form acidic proteoglycan-producing colonies. Thus, it was concluded that MV, but not AB or Exo, has pro-chondrogenic activity.

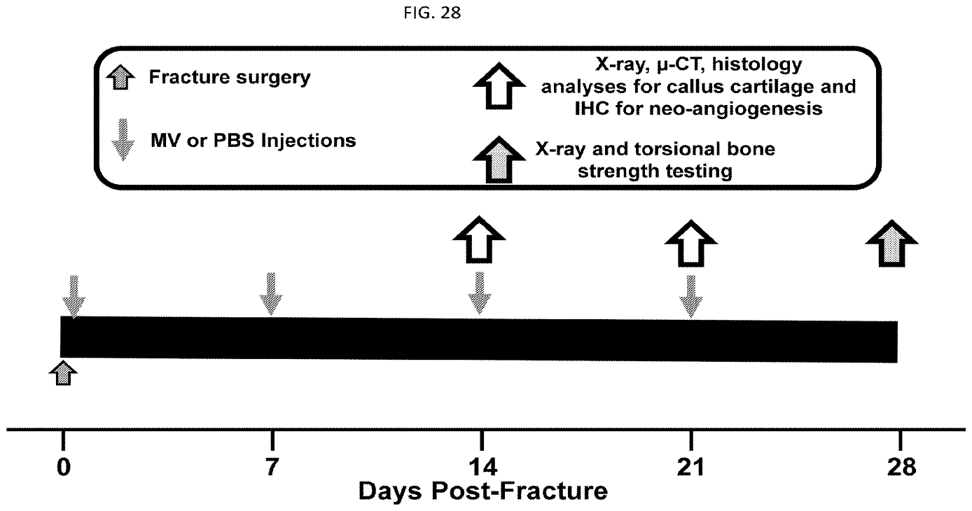

Direct injections of osteoclast-derived MV into fractures accelerated endochondral repair of femoral fractures. Long bone fractures are healed through endochondral ossification that requires chondrogenesis and osteogenesis. Accordingly, the findings that osteoclast-derived MV has both chondrogenic and osteoanabolic activities is important because osteoclast-derived MV can be used as a therapeutic agent to promote endochondral repair of fractures. Thus, it was determined if weekly injections of osteoclast-derived MV into fracture calluses would accelerate the healing of a simple transverse femoral fracture [created by three-point bending technique] in 12-weeks-old male C57BL/6J mice. Briefly, a stainless-steel intramedullary pin (0.56-mm diameter) was surgically implanted prior to fracture to stabilize the femur. A transverse midshaft fracture was then introduced by a controlled blunt impact using Instron servohydraulic tester. This approach produces consistent fractures with minimal comminution that permits both an accurate assessment of repair and humane healing. One-day after the fracture, the untreated control group of 3 mice received direct injection of PBS into the hematoma with the aid of a fluoroscope. [PBS was used as the control, because MV is redissolved in PBS]. The treatment group of 3 mice received weekly injections of a dosage (~45 μg MV protein in 15 μl) of MV isolated from pooled CM of RAW-OC. The healing was first monitored by Faxitron X-ray, which shows that fracture gap (indicated by arrows) in the MV-treated fractures was no longer apparent after 28 days, while the gap remained evident on day 21 and 28 fractures in PBS-injected group of mice (FIG. 15A). The experiment was repeated for 14- or 21-day healing. The fracture healing ability of osteoclast-derived MV was confirmed by increased mineralization of soft calluses to form the low-density woven bone (shown in dark gray—to distinguish it from the high density native cortical bone) at 21 (FIG. 15B) and 28 days (FIG. 15C) and by increased callus cartilage area (stained in red) at 14 days (FIG. 16A). Neo-angiogenesis is important for the facilitation of endochondral bone repair. The MV treatment increased neo-angiogenesis in fracture callus at 21 days, as reflected by drastic increases in IHC staining of vWF (stained in brown) in the soft callus (FIG. 16B). Consistent with pro-angiogenic action, treatment of endothelial cells with osteoclastic MV increased expression of endothelial marker genes (FIG. 17).

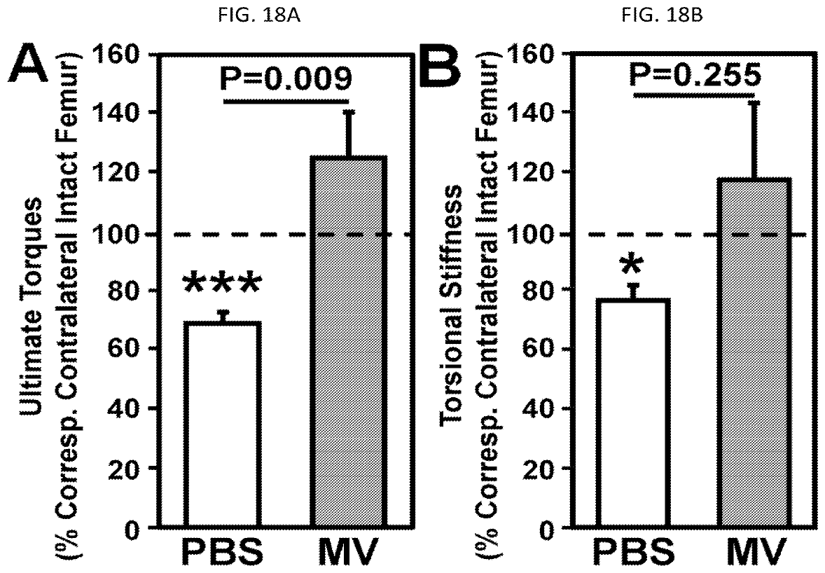

Next, it was evaluated whether the MV treatment would increase bone strength. Torsional mechanical strength testing method was used because it is the most robust method with the smallest error for assessing fracture healing, when compared to three-point bending, four-point bending, cantilever bending, or axial loading, as it is independent of both rotational alignment and embedding uncertainties. Both ultimate torques (FIG. 18A) and torsional stiffness (FIG. 12B) [shown as % of corresponding contralateral intact femur] were greater in MV-treated fractures than in PBS-treated fractures, but the increase in torsional stiffness did not reach a significant level, presumably due to the small group sizes (n=4-6). Together, the foregoing results provide evidence of an enhancing role for osteoclast-derived MV in the endochondral repair of skeletal fractures.

EfnB2 is an attractive candidate for being an important osteogenic component of osteoclast-derived MV. The osteogenic, chondrogenic, and angiogenic activities could be mediated by multiple component(s) or a single, multiple functional, component of osteoclast-derived MV. These components are most probably cytoplasmic membrane-associated growth/differentiation factors or ligands of their receptors. The osteoclastic cytoplasmic membrane-associated osteoanabolic factors further evaluated are Rank, and EfnB2. Rank is the primary osteogenic component in AB fraction released by apoptotic osteoclasts to mediate the local coupled bone formation. Accordingly, apoptotic osteoclasts released large amounts of osteogenic RANK-containing AB, which binds to RANKL on cell surface of osteoprogenitors and triggers the reverse signaling of RANKL to exert osteogenic actions on osteoblasts. When compared to other EV fractions of apoptotic osteoclasts or AB fraction of living osteoclasts, the AB fraction of apoptotic osteoclasts had the highest level of Rank and osteogenic activity, indicating that Rank is a major osteogenic component of AB of apoptotic osteoclasts. It was also assessed whether Rank is an important osteoanabolic component of osteoclastic MV by comparing the relative amounts of Rank in MV and AB fractions from the same pooled CM of RAW-OC by Western immunoblots (FIG. 19). MV, unlike AB, has very low level of Rank. The more salient finding, however, is that despite the substantial amounts of Rank in AB, AB did not significantly increase ALP activity (index of osteoblast differentiation) on osteoprogenitors (FIG. 10B). It also did not promote chondrogenic activity in chondrocytes (FIG. 14). These findings provide evidence that Rank is not a major osteoanabolic component in osteoclastic MV.

Regarding the EfnB2, EfnB2 on the plasma membrane of osteoclasts binds the EphB4 receptor on the cell surface of nearby osteoprogenitors to trigger the EfnB2-EphB4 bidirectional signaling, which on the one hand suppresses osteoclastic resorption through activation of the reverse signaling of EfnB2 in osteoclasts and on the other hand upregulates the EphB4 forward signaling in osteoblasts to stimulate osteoblast maturation and activity. However, this bidirectional signaling mechanism requires direct cell-cell contact between osteoclasts and osteoprogenitors. Given the fact that very few osteoblasts or progenitors are present at the remodeling site that could make direct contact with the resorbing osteoclast, this cell-cell contact-dependent bidirectional mechanism is unlikely a major mechanism for the coupled bone formation under normal situations. It also could not be responsible for the soluble osteoanabolic activity of osteoclasts. However, Western blots show that osteoclastic cells secreted into CM substantial amounts of soluble forms of full-length EfnB2 (FIG. 19). Treatment with EfnA4-fc or PTH increased its releases each by ~75% and the large majority of the released EfnB2 was associated with the MV fraction. As an initial test of the possibility that EfnB2 is a candidate for an osteoanabolic component, it was tested whether the osteogenic activity of CM or that of MV could be blocked by pretreatment with anti-EfnB2 antibody. Indeed, pretreatment of CM released by the PTH-treated calvarial bone (FIG. 20A) or osteoclast-derived MV (FIG. 20B) with a neutralizing anti-EfnB2 antibody reduced their ability to stimulate the BrdU incorporation in MC3T3-E1 cells each by >75%. These findings provide evidence that during bone remodeling osteoclasts release soluble EfnB2-containing MV (EfnB2-MV), which migrates to trailing osteoprogenitors and acts unidirectionally to activate their EphB4 forward signaling to promote local coupled bone formation.

If EfnB2 is indeed an important osteogenic component, MV of osteoclasts deficient in EfnB2 expression should have substantially lower osteogenic activity. To test this prediction, a lenti-EfnB shRNA vector was generated via transient transfection of the Mission® murine EfnB2 shRNA expression plasmid (MilliporeSigma) in HEK293 cells. Briefly, HEK293 cells at 80% confluence were transfected with lenti-EfnB2 shRNA (or lenti-control shRNA) vector at a ratio of 3:1 in 500 μl medium along with VSVG envelope plasmid and Pax2 helper plasmid using Fugene® 6 transfection reagent (Promega) for 48 hr. Viral particles-containing CM was collected, filtered, diluted 2 to 4-fold, and added to RAW cells. After 24 hr, the transduced RAW cells were treated with RANKL to form RAW-OC. Transduction with EfnB2 shRNA reduced EfnB2 mRNA expression in RAW cells by ~80% and protein level in MV by ~75%. Consistent with the suppressive action of the reverse signaling of EfnB2 on osteoclast maturation, the shRNA-mediated suppression of EfnB2 enhanced RAW-OC maturation as reflected by ~30% increase in TRAP activity (FIG. 21, left). Importantly, suppression of EfnB2 expression reduced the ability of MV to increase ALP activity in MC3T3-E1 cells (FIG. 21, right).

Next, it was determined if overexpression of EfnB2 in RAW-OC would enhance the osteogenic activity of its MV. A lenti-EfnB2-GFP vector was generated by subcloning the ORF of EfnB2-C-GFP of pCMV-3-EfnB2-C-GFPSpark plasmid (USA Sino Biological) into a Puro® gene-containing lentiviral vector under control of CMV promoter (Addgene) through transient transfection of this vector plasmid in HEK293 cells. [GFP was added to the C-terminus of EfnB2 to help identification and quantification of the overexpressed EfnB2]. A lenti-GFP vector was also generated as the control. RAW cells were transduced with these lentiviral vectors and treated with RANKL for 6 days to convert to RAW-OC. The MC3T3-E1 cells treated with the EfnB2-overexpressing MV had ~3- and 2-fold greater cell number (FIG. 22A) and ALP activity (FIG. 22B), respectively, than those treated with the control MV. MV isolated from CM of transgenic RAW-OC overexpressing EfnB2 also enhanced the ability of MV to induce formation of mineralized nodule colonies compared to control MV of RAW-OC transduced with the lenti-GFP virus as shown in FIG. 22C. The results show that EfnB2 in the form of MV is an important soluble osteogenic factor released by living osteoclasts.

The osteogenic activity of EfnB2 is mediated via its binding to the EphB4 receptor on osteoprogenitors. Accordingly, MC3T3-E1 cells were pretreated with a blocking anti-EphB4 antibody for 1 hr prior to the addition of osteoclast-derived MV to investigate if blocking the EphB4 forward signaling would abolish the osteogenic activity of MV. Consistent with past findings that the EphB4 forward signaling is a survival/anti-apoptotic pathway, the pretreatment with anti-EphB4 antibody caused large reduction in cell number, increased apoptosis, and reduced BrdU incorporation in MC3T3-E1 cells (FIG. 23A). The anti-EphB4 pretreatment also reduced cellular response to MV with respect to BrdU incorporation by 40% (FIG. 23A) and cellular ALP activity by 80% (FIG. 23B).

There is literature evidence that the soluble EfnB2-mediated EphB4 forward signaling may contribute to chondrogenesis and angiogenesis, during endochondral repair of fractures. Accordingly, the fracture injury upregulated EphB4 expression in hypertrophic chondrocytes in soft callus and osteoblasts/osteocytes in bony callus during the first two weeks post-injury. Conditional overexpression of Ephb4 in Col1-expressing cells enhanced healing of femoral fractures by promoting chondrogenesis and mineralization in the callus and facilitating the 4 phases of endochondral fracture repair. The expression of EfnB2 in pre-hypertrophic chondrocytes, osteoblasts, and osteocytes was upregulated during the first week of fracture healing. Conditional deletion of Efnb2 in Col2-expressing cells delayed fracture healing as a result of reduction in osteo- and chondro-progenitors and VEGF-induced vascular formation in the periosteum and invasion front of the callus. Consistent with an anabolic action of soluble EfnB2 on chondrocytes, it was found that treatment with chondrocytes with EfnB2-fc upregulated expression of chondrogenic genes but not type I collagen (FIG. 24).

To test if EfnB2 on osteoclast-derived MV is important for promoting fracture healing, an experiment was performed in male C57BL/6J mice to compare the therapeutic effect of weekly injections with EfnB2-deficient osteoclast-derived MV (n=4), EfnB2-sufficient osteoclast-derived MV (n=5), or PBS (n=6) on the formation of bony calluses at 14 days of healing. EfnB2-deficient osteoclast-derived MV and EfnB2-sufficient osteoclast-derived MV were isolated from pooled CMs of lenti-EfnB2 shRNA- and lenti-shRNA control-transduced RAW-OC, respectively. In vivo X-ray show substantially less bone minerals (i.e., more opaque) in soft callus of EfnB2-deficient osteoclast-derived MV- or of PBS-treated fractures compared to that of EfnB2-sufficient osteoclast-derived MV-treated fractures (FIG. 25A), which is confirmed by μCT analysis (FIG. 25B). These findings demonstrate that EfnB2 in osteoclast-derived MV is important for promoting the fracture healing process.

It has been shown that the EfnB2/EphB4 signaling also has a regulatory role in the VEGF-mediated neo-angiogenesis in wound healing and tumor growth. The EfnB2-mediated activation of the EphB4 forward signaling in dental pulp stem cells upregulates angiogenesis of endothelial cells. EfnB2 and EphB4 are highly expressed in endothelial cells of developing arteries and veins, respectively, and they are important regulators of vascular development. Moreover, mouse embryos lacking EfnB2 or EphB4 exhibit lethal defects in early angiogenic remodeling. The EphB4 forward signaling has been shown to have important regulatory roles in angiogenesis. Thus, the involvement of the EfnB2 component of osteoclast-derived MV will be investigated in local regulation of chondrogenesis and/or neo-angiogenesis during fracture repair.

Design and methods. Animals are randomized to minimize bias. Both male and female mice are used in experiments, and data analyzed for potential sex differences. The specificity of each commercial antibody used in this study is confirmed by specific blocking with respective blocking peptides prior to use. Data are reported with transparency without bias.

EfnB2 floxed mice (B6. 129S7-Efnb2tm2 And/J, Cat #006042) and EphB4 floxed mice (B6.129JS7-Ephb4tm1 And/J, Cat #006044) are available from the Jackson Lab. The β-actin-luc transgenic mice (B6.Cg-Tyrtm1 Arte (Actb-luc) 46Xen, Cat #11977) is available from Taconic Biosciences. Antibodies to be used are commercially available.

Data and power analyses. The sample size for various endpoints of bone phenotype is determined by power analysis based on standard deviation estimates from similar studies. The group size of “n”=8 animals per group will detect a difference of 20% with a power of 0.80 in various histological parameters. For μ-CT trabecular bone mineral structure and density, we should be able to detect a 10% and 5% difference at p<0.05 with a power of 0.90 and 0.94, respectively, with “n”=8 animals. For bone cell mitogenic activity and ALP assays, “n”=6 will detect difference of 10% at p<0.05 with a power of 0.90. With respect to torsional mechanical testing, the power analysis based on the data on torsional stiffness indicates that the group size of “n=11” will have an α=0.05 and β=0.2 (i.e., a power of 0.80).

Statistical analysis is performed using Systat11 software (Richmond, CA). Normal distribution and homogeneity of variance are first confirmed by the Levene's test. Data with non-normal distribution are analyzed by the nonparametric Kruskal-Wallis test followed by Mann-Whitney U-test method or Wilcoxon Signed-Rank test among various experimental group and time points. For data sets showing normal distribution, comparison of differences between two variables is done with the two-tailed, two-sample with equal variances independent t-test. Comparison of multiple groups is performed with analysis of variance (ANOVA) with Tukey's HSD post-hoc testing. Simple or multivariant correlation analyses and linear regression analyses are performed to assess associative relationship among the various parameters. The differences are significant when p<0.05.

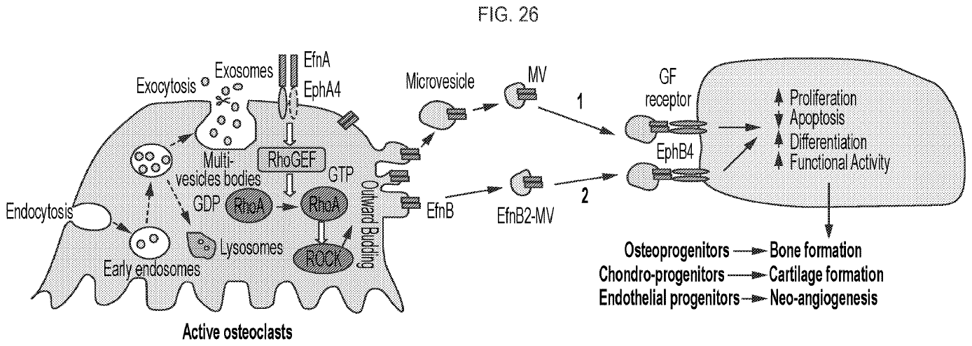

General experimental strategy. This study seeks to evaluate the mechanism of the EphA4-mediated osteoclastic regulation of local coupled bone regeneration. In our working model (FIG. 26), the EphA4 forward signaling in osteoclasts is an important regulatory mechanism for the genesis and release of MV. This part of the model is based on findings that 1) the EphA4 forward signaling in osteoclasts upregulates the RhoA/ROCK signaling, 2) the RhoA/ROCK signaling is important for the biogenesis and cellular release of MV, 3) inhibition of RhoA activity in osteoclasts by CCG-1423 suppressed basal and EphA4-dependent releases of bone cell mitogenic activities, and 4) mice with deficient Epha4 expression in osteoclasts showed defective local coupled bone formation. The core of this working model, which is the focus of this investigation, is important to the molecular mechanism by which the released osteoclast-derived MV promotes local bone regeneration. The principal concept (identified as 1) speculates that osteoclast-derived MV contains one or more membrane-associated growth/differentiation factors (GF) or ligands for their receptors. Upon its release, osteoclast-derived MV migrates to nearby progenitors of osteoblasts, chondrocytes, or endothelial cells, binds to cognate receptor(s), and triggers their signaling to promote their proliferation, differentiation, and activity, which in turn increases bone formation, cartilage synthesis, and vascularization, respectively. These concerted actions, which occur spatially and temporally with each other, would lead to the eventual regeneration of bone tissues with structural and mechanical properties indistinguishable from those of native bone. There is an alternative concept (identified as 2), which postulates that EfnB2 is an important osteoanabolic component of MV. It speculates that the released EfnB2-containing MV migrates to nearby osteo-, chondro-, and/or endothelial-progenitors to activate the EphB4 forward signaling on respective target cells, leading to increased endochondral bone formation. The rationale for the alternative concept is as follows: 1) osteoclast-derived MV contains EfnB2, 2) EfnB2 has anabolic activity on osteoprogenitors, 3) pretreatment of osteoclast-derived MV with anti-EfnB2 antibody abrogated its osteogenic activity, 4) pretreatment of osteoprogenitors with anti-EphB4 antibody blocked the osteogenic activity of MV, 4) EfnB2-deficient osteoclast-derived MV had reduced osteogenic activity, 5) EfnB2-overexpressing osteoclast-derived MV showed greater osteogenic activity, 6) soluble EfnB2-fc stimulated chondrogenesis, and 7) EfnB2 has an important anabolic role for in chondrogenesis and angiogenesis.

The principal concept will be evaluated with three sets of experiments. The first set is to establish an optimal local injection regimen and an optimal dose range for osteoclast-derived MV. It will first be determined the duration-of-stay of the injected MV inside the fracture, because this information is important for the proper design of subsequent experiments to determine optimal injection frequency and duration between injections. The efficacy of single vs. multiple injections on fracture healing will be compared using the duration-of-stay information to decide on an optimal duration between injections for multiple injections. Then, the optimized injection regimen will be utilized to determine an optimal dose range. The primary endpoints for these experiments are formation/remodeling of callus cartilage and bony tissues, as replacement of callus cartilage with bony tissues is an important index of bony bridging of the fracture gap. The second set is to ascertain that an optimized injection regimen of osteoclast-derived MV would promote fracture healing and restore mechanical strength of the healed bone in both male and female mice. This experiment is important and relevant since the full return of bone strength is perhaps the most clinically meaningful outcome for fracture healing. Previous clinical studies indicate female sex being a major risk factor for compromised bone healing in elderly women. Studies in 12-months-old rats and 8- to 12-weeks-old C57BL/6 mice also reveal that basal fracture healing in females is less robust than that in males. Thus, this study will be performed in both male and female mice to determine if there are also sex differences in the fracture healing response to MV. The third set is to perform histological evaluation of the time-dependent effects of MV treatment on relevant skeletal cell types within the fracture, especially during formation and remodeling of cartilaginous calluses and bony calluses, with special attention to osteoblasts, osteoclasts, and chondrocytes. Information about of the types of cells and at each stage of the fracture repair process on which MV acts to yield osteoanabolic effects is important for the overall understanding of the cellular mechanism by which osteoclast-derived MV accelerates fracture healing. It may also confirm that osteoclast-derived MV promotes cartilaginous callus formation and neo-vascularization at early phases and bony callus formation at later phases of fracture healing.

It is important to know the identity of the important osteoanabolic component(s) of osteoclast-derived MV, since information about the identity of important osteoanabolic components is important for the proper use and improvement of future osteoclast-derived MV-based therapeutic strategies. The “candidate functional testing” approach will be used to evaluate the alternate concept that EfnB2 is an important osteoanabolic component of osteoclastic MV. It will be determined: 1) if treatment of osteoblasts, chondrocytes, endothelial cells with an effective dose of osteoclast-derived MV upregulates the forward signaling of EphB4, 2) if MV of EfnB2-deficient osteoclasts would show diminished osteogenic, pro-chondrogenic and pro-angiogenic activities, in vitro, 3) if MV of osteoclasts with EfnB2 overexpression enhances its in vitro osteogenic, chondrogenic, and angiogenic activities, 4) if osteoprogenitors, chondrocytes, and endothelial cells deficient in EphB4 expression show diminished in vitro osteogenic, chondrogenic, and angiogenesis responses to osteoclastic MV, and 5) if local injections of EfnB2-deficient MV impedes, and EfnB2-overexpressing MV accelerates, fracture healing in vivo.

To determine an optimal injection regimen and dosage for the osteoclast-derived MV strategy to promote fracture healing using a simple transverse femoral fracture model. For these studies, the regimen for local osteoclast-derived MV injections will be determined and optimized. Other than the injection route, which is direct injections into fractures, the important elements of an optimized administration regimen are 1) frequency (e.g., a single or multiple injections), 2) duration between injections if multiple injections are needed, and 3) dose range. The duration-of-stay of osteoclast-derived MV at the fracture after its local injection will be determined. Information about the duration-of-stay of MV within fracture callus is then used to determine whether multiple injections are needed, and if so, an appropriate injection frequency and duration between injections. An optimized injection regimen will be used to determine an optimal dose range of osteoclast-derived MV. Male mice will be used for these experiments for the following reasons: 1) basal fracture healing in male C57BL/6J mice is more robust compared to that in female mice. Thus, the use of male mice would increase the odds for bigger and more significant healing response; 2) Men tend to have more long bone fractures than women before age of 50; 3) Men usually have fractures at non-vertebral sites with higher bone density; and 4) The male veteran population has higher risks for long bone fractures than the female veteran population. Next, an optimized injection regimen will be determined which will yield clinically meaningful results, e.g., increases in mechanical strength and acceleration of bony bridging of fracture gap. This set of experiments also determines if there are gender-related differences in the response to osteoclast-derived MV on fracture healing by comparing important endpoints of fracture healing in male mice with those in female mice. These experiments will be followed by determining the cellular mechanism(s) by which osteoclast-derived MV acts to accelerate fracture healing. Histological and IHC approaches will be applied to identify the type and activity of cells within the callus that respond to MV throughout the four stages of the endochondral bone repair process. Histological and IHC analyses of time-dependent effects of MV treatment will be performed on osteoclasts, osteoblasts, and chondrocytes throughout the course of the fracture healing process.

Experimental approach. A simple transverse femoral fracture model will be used because weekly injections of osteoclast-derived MV into fractures promoted fracture healing in this model (FIGS. 15-18). Osteoclast-derived MV is isolated from pooled CM of primary osteoclasts generated from marrow-derived precursors with 50 ng/ml m-CSF and 100 ng/mL RANKL by differential ultracentrifugation. This experimental approach is divided in three parts. The first part, which has 3 experiments, is to determine an optimal injection regimen of osteoclast-derived MV. The first experiment is to determine the duration-of-stay of osteoclast-derived MV at fracture callus after local injection. To monitor location of the injected MV in vivo, the Perkin-Elmer IVIS Spectrum CT system will be used because it provides high-sensitivity in vivo imaging of fluorescence and bioluminescence in small animals. The IVIS Spectrum CT system shows higher sensitivity for bioluminescence than fluorescence. Luciferase-expressing MV will be used as the tracer because luciferase-expressing macrophages has been used as the tracer to determine the duration-of-stay of the injected macrophages in knee joints after intraarticular injections. The β-actin-luc transgenic mice are available from Taconic Biosciences. Because osteoclast-derived MV contains large amounts of β-actin (FIG. 19), MV of osteoclasts derived from these transgenic mice is an excellent tracer for in vivo monitoring of the fate of MV after injections with high sensitivity. Briefly, osteoclast progenitors are isolated from β-actin-luc transgenic mice and treated with m-CSF/RANKL to generate osteoclasts. β-actin-luc-expressing MV is isolated from pooled CMs and is injected into femoral fractures of a group of 8 male C57BL/6J mice at one day post-fracture. On the day of MV injection and on day 1, 3, 5, 10, 14, and 21 days, each mouse receives an i.p. injection of luciferin under anesthesia at 20 min before 3D imaging is taken. The relative intensity and location of β-actin-luc-expressing MV in live mice is monitored and recorded with the IVIS Spectrum CT System. Hair around the fracture is removed prior to imaging because animal hair blocks, absorbs, and scatters light. Bioluminescence imaging at fracture callus at each time point is recorded and compared. The remaining bioluminescence that is <5% of the original injected amounts will be considered being functionally insignificant. Changes in location and intensity of bioluminescence with time will provide important information about the duration-of-stay of injected MV in the callus.

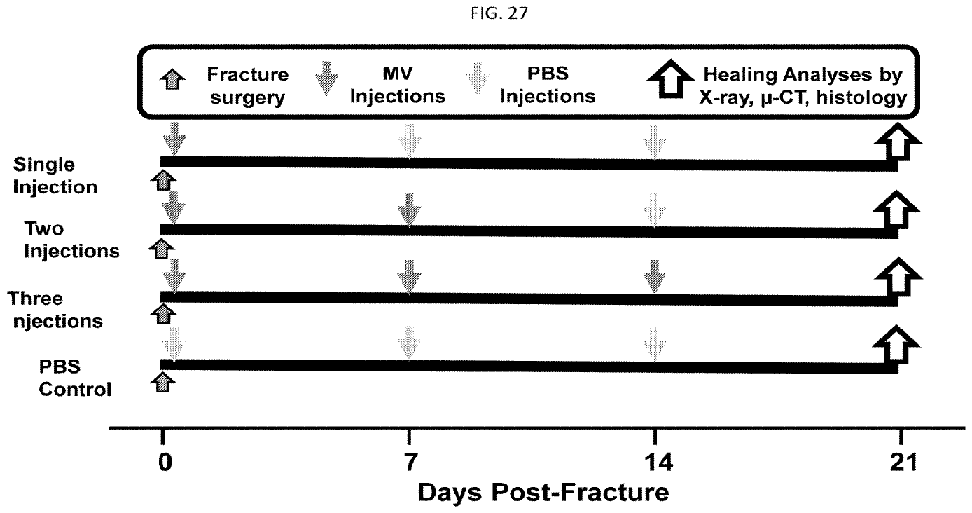

The next experiment determines if a single injection of osteoclast-derived MV is sufficient or multiple injections are required to produce an optimal fracture healing response. The strategy is shown in FIG. 27. [The actual duration interval between injections is determined by the results of duration-of-stay experiment.]. Male mice will be used and endpoints will be measured at a single time point. A 21-day time point was chosen because data described herein indicate that differences in callus cartilage remodeling between MV group and PBS control group seem to be the biggest at this time point. Additional time points may be added if needed. Assessment of fracture healing is limited to measurements of callus cartilage and bony tissue areas in fractures by μCT and histology, since formation and remodeling of calluses are generally considered as indices of fracture healing. There are 4 groups of 8 male C57BL/6J mice each: 1) single injection group (receiving osteoclast-derived MV on day 1 and PBS on day 7 and 14), 2) two injections group (receiving osteoclast-derived MV on day 1 and 7 and PBS on day 14), 3) three injections group (receiving osteoclast-derived MV injections on day 1, 7, and 14), and 4) control group (receiving three injections of PBS on day 1, 7, and 14). Injections into fractures are done with the aid of a fluoroscope. Briefly, on day 1, 7, 14, and 21 days, evidence for fracture healing in each mouse is monitored in vivo in live animals by Faxiton X-ray. On day 21, the mice from each group are euthanized. Fractures are examined for relative amounts of callus cartilage and trabecular bones by histology and μ-CT (Gregory, C. A., et al. (2004) Anal Biochem 329, 77-84). Briefly, the fractured femur is harvested, the fixation pin carefully removed, and fixed in 4% paraformaldehyde. Each fractured bone is then analyzed by μ-CT using a Scanco vivaCT 40 μCT system with a voxel size of 10.5 μm. Each sample is contoured around the callus circumference. The chosen thresholds distinguish the newly formed lower-density woven bones (220 to 570 mg/cm³) in fracture calluses from native higher-density cortical bones (570 to 1000 mg/cm³). The μCT parameters of lower-density woven bone (connectivity density, BV/TV, BMD, Tb.N, Tb.Th., and Tb.Sp.) are determined and compared among the test groups. After μCT measurements, the fractured bones are decalcified, embedded into paraffin wax, sectioned to 5 μm in thickness, and kept at 4° C. until staining with Safranin O for cartilage (Lau, K. H., et al. (2013) Bone 53, 369-381). Callus cartilage area is reported as percentage of total callus area measured with the OsteoMetrics system. Measurements are performed in a blinded fashion to avoid unintended biases. Comparison of the relative amounts of cartilage and bony tissues at the fracture callus among treatment groups will provide information as to whether multiple MV injections are important for an optimal healing effect.

The next experiment is to determine an optimal dose range using the optimized injection regimen. A single preparation of osteoclast-derived MV will be used for each dosage experiment and it will be repeated with at least two other preparations to confirm reproducibility. Total MV protein content will be used as a measure of dosage. Aliquots of each preparation are saved. When information about the identity of active ingredient(s) is available, the amounts of active component(s) (e.g., EfnB2) in each MV preparation are determined by ELISA and an optimal dose range is then converted back to one based on the amount of the active component. The results described herein show that weekly injections of 45 μg (in 15 μL) of osteoclast-derived MV protein into fracture of a mouse of ~25 g (or ~2 mg MV protein/kg body weight) promoted fracture healing (FIGS. 15, 16, and 18). Thus, the dose ranging from 0 (PBS), 0.5, 2, to 5 mg MV protein/kg body weight was initially tested. Additional doses are added. Fractures are examined for healing as described herein. Analyses of the results of these experiments will be used to determine an optimal injection regimen and dose range.

The second part seeks to demonstrate that the osteoclast-derived MV treatment, using the optimized injection regimen and dosage, yields clinically meaningful benefits in both male and female C57BL/6J mice. The primary endpoint is the restoration of bone strength, which is determined by the torsional bone strength test on day 28 of healing. [This time point is chosen based on the findings shown in FIG. 18]. The secondary endpoint is evidence for an accelerated bony bridging of the fracture gap. [Bony bridging of fracture gap is determined by μ-CT analysis as well as histology of cartilage and bony tissues at fracture site on day 14 and 21]. The third endpoint is neo-angiogenesis. There are 4 groups of 12 weeks old mice: 1) male PBS group-27 male mice receiving PBS injections, 2) male MV group—27 male mice receiving osteoclast-derived MV injections, 3) female PBS group—27 female mice receiving PBS injections, and 4) female MV group—27 female mice receiving osteoclast-derived MV injections. The established optimized injection regimen and dosage are used. The experimental strategy is shown in FIG. 28. Fracture surgery is performed on the right femur of each mouse on day 0. On day 1, 7, 14, and 21, the fracture site of the mice of the PBS groups receives PBS injections and that of MV groups receives injections of an effective dose of osteoclast-derived MV. Faxitron X-rays are taken of each mouse in vivo on day 1, 7, 14, 21, and 28. On day 14 and 21, 8 randomly selected mice from each group are euthanized. Fractured bones are harvested, cleaned, pin removed, fixed with paraformaldehyde, and subjected to ex vivo X-ray, μ-CT and bone histology analyses for callus cartilage and bone formation and remodeling (as described herein) as indices for bony bridging of the fracture gap. Additional time points are added if they help better define the timing of bony bridging. Serial paraffin sections are analyzed for newly formed blood vessels by IHC staining of von Willebrand's Factor (vWF) [or CD31] to assess effects of MV on neo-angiogenesis at fracture calluses. Comparison of the relative amounts of cartilage and bony tissues at fracture calluses between the various time points and among the four groups by 2-way ANOVA should yield important information as to whether osteoclast-derived MV accelerates bony bridging of fracture gaps and whether there are sex differences in the response to osteoclast-derived MV. On day 28, the remaining 11 mice from each group are euthanized and fractures examined for bone strength recovery by torsional mechanical bone strength testing. Briefly, the epiphyses of unfixed fractured and contralateral intact femur of each mouse are cast into a dental resin that adheres to the bone and allow each end of the femur to be secured in the opposing jaws of the torsional tester. Torsional force is applied at 1°/s until failure. Torsional stiffness (in N-mm/°) [calculated from the linear portion of the force vs angle curve prior to the yield point] and ultimate torque (N-mm), two important measures of torsional bone strength, are determined for each femur. The relative return of torsional bone strength to pre-injury strength of each fractured bone is shown as the ratio of torsional stiffness of fractured bone to that of contralateral intact bone. A ratio of 1.0 indicates full return of its torsional stiffness. An increased return of torsional stiffness confirms an enhanced fracture healing.

The third part is a time course study to evaluate the time-dependent effects of osteoclast-derived MV on the number and functional activities of osteoclasts, osteoblasts, chondrocytes inside fracture calluses during the healing process. This analysis is done exclusively by histology and IHC. Briefly, fracture surgery is performed on two groups of 48 male C57BL/6 mice of 12 weeks of age each. On 1 day after surgery (and an appropriate interval thereafter), the control group receives PBS (15 μl) injection into fractures, whereas the MV group receives a 15 μl of an effective dose of osteoclast-derived MV. On day 5 (hematoma phase), 7 (soft callus phase), 14 (early bony callus phase), 21 (mid bony callus and beginning of bone remodeling phase), 28 (late bony callus phase and mid remodeling phase), and 35 (late remodeling phase), randomly selected 8 mice from each group are sacrificed and fractured femurs excised, cleaned, fixed with 10% buffered formalin for 48 hrs, decalcified with 10% EDTA, dehydrated with ascending alcohols, and embedded in paraffin wax. Thin sagittal sections are cut with a microtome. Serial sections are stained with H&E (for general cell morphology), van Gieson's stain (for collagen fibers), H&E plus alcian blue (highlight cartilage), picrosirius red (for type I and type III fibers), ALP (for osteoblasts), TRAP (for osteoclasts), and alcian blue/orange G (for chondrocytes). Serial sections are also IHC stained for osteocalcin (for osteoblast activity), RunX2 (for osteoprogenitors), and CD31 (for endothelial cells/blood vessels). The location and area of cartilage, woven bone, blood vessels, and the number of blood vessel per bone surface of osteoclasts and osteoblasts are determined. Analysis is done in a blinded fashion in 2 to 3 serial sections to obtain average values. Comparison of time-dependent changes in these parameters between MV-treated and PBS-treated control fractures provides insights into the effects of osteoclast-derived MV on various cellular aspects of the fracture healing process. An integrative analysis of these changes may help to delineate the cellular mechanism by which osteoclast-derived MV regulates each of the four phases of the fracture healing process.

It is expected that 1) an optimal injection protocol and dose range of osteoclast-derived MV for fracture repair can be determined; 2) multiple injections of osteoclast-derived MV are needed for optimal effects; 3) osteoclast-derived MV treatment can effectively accelerate bony bridging of fracture gaps and increase the return of torsional bone strength of fractured bones in both genders; and 4) osteoclast-derived MV accelerates three of the four phases of the fracture repair process (fibrocartilaginous callus formation, bony callus formation, and bone remodeling phases) with anabolic effects on osteoblasts and chondrocytes without significant effects on osteoclasts.

Differential ultracentrifugation can cause vesicles disruption, aggregation, and co-localized with other non-MV bioactive components. In some aspects, a sucrose gradient ultracentrifugation can be used. In some aspects, the presence of luciferase by IHC will be determined at each time point. The findings in FIG. 18 indicate that the osteoclast-derived MV treatment should accelerate the return of torsional bone strength. However, because the torsional strength of untreated (PBS) fractures have already returned to 70-80% of that of contralateral intact bone on day 28, this time point might not offer sufficient sensitivity for detecting statistically significant beneficial effects. If so, we will assess bone strength on day 21, during which the healing effect on untreated fractures should be much smaller such that it would provide a better chance for significant differences. If a single injection is insufficient, it will be tested if the use of a hydrogel scaffold as carrier would prolong duration-of-stay of the MV in future. Several injectable hydrogels have been used in fracture healing.

To determine if EfnB2 is an important osteoanabolic component of osteoclast-derived MV. The data described herein demonstrate that EfnB2 in osteoclast-derived MV may be an important osteoanabolic component and useful as a therapeutic strategy. “The candidate testing strategy” will be used to determine whether EfnB2 in osteoclast-derived MV meets the criteria. Thus, EfnB2 in osteoclast-derived MV will be evaluated as an important osteoanabolic component. For example, the chosen candidate testing strategy tests the following: 1) osteoclast-derived MV treatment should upregulate the EphB4 forward signaling in target cells in vitro; 2) MV of EfnB2-deficient osteoclasts should have reduced osteoanabolic activities in vitro; 3) MV of EfnB2-overexpressing osteoclasts should show enhanced osteoanabolic activities in vitro; 4) osteoblasts, chondrocytes, and endothelial cells of EphB4-deficient mice should show diminished osteoanabolic responses to osteoclast-derived MV in vitro; and 5) EfnB2-deficient MV should reduce, and EfnB2-overexpressing MV should enhance, the fracture healing activity of MV in vivo. The data provided herein used MV derived from RAW-OC, but these experiments will use MV of primary osteoclasts.

Experimental approach. To test EfnB2 as a therapeutic strategy, five experimental strategies will be carried out. The first strategy is to determine if treatment of osteoblasts, chondrocytes, and endothelial cells with an effective dosage of osteoclast-derived MV would indeed result in upregulation of the forward signaling of EphB4 in each target cell type (e.g., osteoblasts, chondroblasts, and endothelial cells). Osteoblasts are isolated by sequential collagenase digestion of bone chips. Primary chondrocytes are isolated from articular cartilage layers of C57BL/6 mice (Stiffel, V. M., et al. (2020) Calcif Tissue Int 107, 576-592). Primary endothelial cells are isolated (Sokol, L., et al. (2021) STAR Protoc 2, 100489). [ATDC5 chondrocytic cells and C166 endothelial cells may also be used if their responses are similar to primary cells]. The activation status of EphB4 forward signaling is determined by measuring pY-987 EphB4 phosphorylation level with Western analysis. Because activation of the EphB4 forward signaling increases EphB4 phosphorylation (e.g., pY-987) that in turn activates Fak, Src, Shc, and RhoA by increasing pY397-FAK, pY416-Src, pY239/240-Shc, pS188-RhoA and pYErk1/2, the phosphorylation levels of these signaling proteins are determined by Western blots. Signaling mediators of unrelated pathways (Jak1, p38 MAPK) are included for comparison. Unlike osteoclastic MV, MV of MC3T3-E1 cells or HEK293 cells did not increase both osteoblastic proliferation and differentiation (FIG. 11A). Thus, MV of MC3T3-E1 cells or that of HEK293 cells may be included in this experiment for comparison.

The second strategy is to determine if MV derived from osteoclasts deficient in expression of EfnB2 would show diminished osteoanabolic activities compared to control MV. The study described herein used the lenti-EfnB2 shRNA (FIG. 21) and blocking anti-EfnB2 antibody (FIG. 20) approaches to show that MV deficient in EfnB2 expression or activity had reduced osteogenic activity. To obtain EfnB2-deficient osteoclasts, isolated from EfnB2 floxed mice (available from the Jackson Labs Cat #006042) osteoclast precursors will be treated with Adenoviral-Cre vector (or emptied adenovirus as control) to delete the EfnB2 gene. The Cre-treated cells are then converted into osteoclasts with the m-CSF/RANKL treatment. EfnB2-deficient and control MV are isolated from pooled CM of Ad-Cre and control Ad-treated osteoclasts, respectively. To confirm EfnB2 deficiency, the EfnB2 level in MV is measured by ELISA. It will be determined if MV of EfnB2-deficient osteoclasts would have reduced osteogenic, chondrogenic, and angiogenic activities in vitro. Osteogenic activities are assessed as follows: Primary osteoblasts are treated with an effective dose of each MV (or PBS as control) for 24 hrs (for proliferation and apoptosis assay). Osteoblast proliferation is assessed by BrdU incorporation. Osteoblast apoptosis is determined with a commercial apoptosis assay kit (e.g., Roche Cell Death Detection ELISA kit). Osteoblast differentiation is evaluated by measuring cellular ALP specific activity, and expression levels of various osteoblastic differentiation marker genes (Col1, Opn, Ocn, Osx, Runx2/Cbfa1, and ALP) by RT-qPCR after 48 hrs or up to 3 weeks of incubation with each MV and PBS. The results are normalized against level of EfnB2 as well as Arf6 or total protein in MV. Osteoblast activity is monitored by the formation of mineralized nodules in osteoblastic cells after 21 days of incubation with MV or PBS in an osteogenic medium (αMEM containing 10% FBS, 10 mM β-glycerophosphate, and 50 μg/mL ascorbic acid). Mineralized nodules are stained with alizarin red, extracted with 10% acetic acid, and absorbance determined at 405 nm. Chondrogenic activity of each MV is monitored by its effect on the proliferation, apoptosis, maturation, and activity of the treated chondrocytes. Primary chondrocytes are treated with each MV or PBS for 24 hrs (for proliferation and apoptosis) and for 48 hrs, 1, 2, and 3 weeks (for differentiation). Proliferation and apoptosis are measured with BrdU incorporation and apoptosis ELISA kits, respectively. Chondrocyte maturation is assessed by expression level of chondrocytic genes (Col2α1, Col10α1, Mmp13, Acan, Sox9, Prg4) by RT-qPCR. Activity is determined by the ability of treated chondrocytes to form acidic proteoglycan-producing colonies, which are stained with alcian blue, eluted with 8M guanidine HCl, and quantitated by measuring its absorbance at 600 nm. Angiogenic activity of MV is determined by comparing their effects on proliferation, apoptosis, differentiation, and functional activity of endothelial cells. Endothelial cell proliferation and apoptosis are determined with BrdU incorporation and apoptosis assay kits, respectively. Endothelial cell differentiation is monitored by the expression levels of endothelial marker genes (VE-cadherin, CD31/PECAM-1, vWF-A2, VEGFR1/Flt-1, VEGFR2/KDR/Flk-1, VCAM-1/CD106) with RT-qPCR. In vitro functional activity is determined by tube formation assay using an in vitro angiogenesis assay kit (MilliporeSigma). Angiogenic activity is determined by the total area of endothelial cell tube formed per area (by OsteoMeasure) per Arf6 (or total MV protein) as well as EfnB2. Comparison of osteoanabolic activities of EfnB2-deficient MV with those of EfnB2-sufficient MV will provide information on whether EfnB2 is an important osteoanabolic activity of MV.

The third strategy is to determine if overexpression of EfnB2 in osteoclast-derived MV enhances their in vitro osteoanabolic activities. A lenti-EfnB2-GFP vector was produced for use to generate EfnB2-overexpressing RAW-OC and show that EfnB2-overexpressing MV significantly enhanced its osteogenic activity in MC3T3-E1 cells (FIG. 22). The EfnB2 overexpression lentiviral vector and the corresponding control lenti-GFP vector will be used to transduce primary osteoclast progenitors. Briefly, osteoclast progenitors at near confluency are transduced with lenti-EfnB2-GFP (or lenti-GFP) as described herein (FIG. 22). Positive stable clones are selected with puromycin, expanded, and cultured with m-CSF/RANKL for 6 days. CM of multiple dishes of lenti-EfnB2-GFP- or lenti-GFP-transduced osteoclasts is each pooled separately and MV is isolated by differential ultracentrifugation. The level of EfnB2 in each preparation is measured by ELISA. The in vitro osteogenic, chondrogenic, and angiogenic activities of EfnB2-overexpressing and control MV is determined as described herein. To determine whether EfnB2 is an important osteoanabolic component, the in vitro osteoanabolic activities of EfnB2-overexpressing MV will be compared with those of control MV.

The fourth strategy is to determine if EphB4-deficient osteoblasts, chondrocytes, and/or endothelial cells show reduced in vitro osteogenic, chondrogenic, and angiogenic responses, respectively, to osteoclastic MV. EphB4 floxed mice (B6.129S7-Ephb4tm1And/J) are available from the Jackson Lab. The approach is to isolate osteoblasts, chondrocytes, and endothelial cells from these floxed mice and transduced with Ade-Cre (or control Ade) vector to delete the EphB4 gene. After confirmation of successful deletion by measuring the EphB4 mRNA and protein expression in the Ad-Cre-treated cells by qRT-PCR and ELISA, respectively, each EphB4-deficient and corresponding control-Ad-treated cell type is treated with an effective dose of osteoclastic MV (or PBS). The osteogenic, chondrogenic, and angiogenic activities, respectively, of EphB4-deficient and control cells are determined and compared as described herein. It is expected that deficient EphB4 expression in target cells should abolish the stimulatory effects of MV on its osteogenic, chondrogenic, or angiogenic activity.

The last strategy is to show if EfnB2-deficient osteoclast-derived MV has reduced, while EfnB2-overexpressing MV has enhanced, fracture healing response compared to respective controls. EfnB2-deficient MV and its control MV are derived from Ad-Cre-treated EfnB2 floxed and control Ad-treated osteoclasts, respectively, as described herein. EfnB2-overexpressing MV and control MV are derived from lenti-EfnB2-GFP- and lenti-GFP-transduced osteoclasts, respectively, as shown in FIG. 22. There are 4 groups of 8 male 12-weeks-old C57BL/6J mice each: 1) EfnB2-deficient MV group; 2) MV from corresponding control group; 3) EfnB2-GFP-overexpressing group; and 4) GFP-expressing control group. The experimental design of the fracture healing study is the same as that for osteoclastic MV as shown in FIG. 26. On day 21, the 8 mice from each group are euthanized and fracture healing are determined by X-ray, μ-CT, and histology as described herein. The fracture healing parameters will be compared between the EfnB2-deficient MV group and its control group and between the EfnB2-overexpressing MV group.

Expected results. The results will provide evidence supporting EfnB2 as an important osteoanabolic component of osteoclast-derived MV. The results are also expected to confirm that EfnB2-deficient MV group of mice will show reduced fracture healing enhancing ability compared to the corresponding control group of mice, and conversely, that MV with overexpression of EfnB2 group of mice will show enhanced fracture healing enhancing activity compared to control.

In some aspects, the nano-LC-MS/MS technology and its proteomic analytical algorisms can be used to identify osteoanabolic proteins within osteoclast-derived MV. Osteoanabolic proteins within osteoclast-derived MV identified can be selected for functional test by strategies as described herein for EfnB2. In some aspects, pooled CM from different preparations of transduced osteoclasts will be used and the EfnB2 expression level in MV by ELISA will be measured to ensure sufficient EfnB2 overexpression before use.

What is claimed is:

1. A composition comprising, one or more liposomes, and a targeting peptide, wherein the one or more liposomes comprise a transmembrane protein, and wherein the targeting peptide is outside one or more of the liposomes.

2. The composition of claim 1, wherein the one or more liposomes is a multilamellar vesicle.

3. The composition of claim 2, wherein the multilamellar vesicle comprises 1,2-dioleoyl-3-trimethylammonium-propane (DOTAP), Dioleoylphosphatidylethanolamine (DOPE), cholesterol, DSPE-mPEG2000, DSPE-PEG2000-MAL, or a combination thereof.

4. The composition of claim 3, wherein the multilamellar vesicle comprises 1,2-dioleoyl-3-trimethylammonium-propane (DOTAP), Dioleoylphosphatidylethanolamine (DOPE), cholesterol, DSPE-mPEG2000, and DSPE-PEG2000-MAL.

5. The composition of claim 4, wherein the multilamellar vesicle comprises 1,2-dioleoyl-3-trimethylammonium-propane (DOTAP), Dioleoylphosphatidylethanolamine (DOPE), cholesterol, DSPE-mPEG2000, and DSPE-PEG2000-MAL in a ratio of 42:15:38:3:2.

6. The composition of claim 1, wherein the transmembrane protein is EfnB2, or a modified or recombinant EfnB2.

7. The composition of claim 1, wherein the targeting peptide consists of DSS DSS DSS DSS DSS DSS (SEQ ID NO: 1), DDDDDDDD (SEQ ID NO: 6), DDDDDDDDDDDDDD (SEQ ID NO: 7). DSS DSS DSS DSS DSS DSS (SEQ ID NO: 8) or KNFQSRSH (SEQ ID NO: 9).

8. The composition of claim 7, wherein the targeting peptide consisting of DSS DSS DSS DSS DSS DSS (SEQ ID NO: 1) and binds to hydroxyapaptide crystals.

9. A pharmaceutical composition comprising the composition of any of the preceding claims.

10. The pharmaceutical composition of claim 9, further comprising at least one pharmaceutically acceptable excipient.

11. A composition comprising, one or more osteoclast-derived microvesicles, and a targeting peptide, wherein the one or more osteoclast-derived microvesicles comprise a transmembrane protein, and wherein the targeting peptide is outside the one or more osteoclast-derived microvesicles.

12. The composition of claim 11, wherein the transmembrane protein is EfnB2 or a modified or recombinant EfnB2.

13. The composition of claim 11, wherein the targeting peptide consists of DSS DSS DSS DSS DSS DSS (SEQ ID NO: 1), DDDDDDDD (SEQ ID NO: 6), DDDDDDDDDDDDDD (SEQ ID NO: 7). DSS DSS DSS DSS DSS DSS (SEQ ID NO: 8) or KNFQSRSH (SEQ ID NO: 9).

14. The composition of claim 13, wherein the targeting peptide consisting of DSS DSS DSS DSS DSS DSS (SEQ ID NO: 1) binds to hydroxyapaptide crystals.

15. A pharmaceutical composition comprising the composition of any of claims 11 to 14.

16. The pharmaceutical composition of claim 15, further comprising at least one pharmaceutically acceptable excipient.

17. A method of treating osteoarthritis or post-traumatic osteoarthritis in a subject, the method comprising administering to the subject a therapeutically effective amount of the composition of any of claims 1 to 8 or 11 to 14 or the pharmaceutical composition of any of claims 9 to 10 or 15 to 16.

18. A method of upregulating chondrogenic gene expression in primary articular chondrocytes, the method comprising administering to a subject in need thereof a therapeutically effective amount of the composition of any of claims 1 to 8 or 11 to 14 or the pharmaceutical composition of any of claims 9 to 10 or 15 to 16.

19. A method of regenerating articular cartilage and subchondral bone structure in a subject, the method comprising administering to the subject in need thereof a therapeutically effective amount of the composition of any of claims 1 to 8 or 11 to 14 or the pharmaceutical composition of any of claims 9 to 10 or 15 to 16.

20. A method of promoting acidic proteoglycan-producing nodule formation in a subject, the method comprising administering to the subject in need thereof a therapeutically effective amount of the composition of any of claims 1 to 8 or 11 to 14 or the pharmaceutical composition of any of claims 9 to 10 or 15 to 16.

21. A method of reducing or reversing synovial hyperplasia at an injured joint in a subject, the method comprising administering to the subject in need thereof a therapeutically effective amount of the composition of any of claims 1 to 8 or 11 to 14 or the pharmaceutical composition of any of claims 9 to 10 or 15 to 16.

22. The method of claim 21, wherein the injured joint is a hand, lower back, knuckle, neck, feet, toe, knee, hip, ankle, shoulder or elbow.

23. A method of reducing inflammation at an injured joint in a subject, the method comprising administering to the subject in need thereof a therapeutically effective amount of the composition of any of claims 1 to 8 or 11 to 14 or the pharmaceutical composition of any of claims 9 to 10 or 15 to 16.

24. The method of claim 23, wherein one or more cytokines in the injured joint of the subject are reduced.

25. The method of claim 24, wherein the one or more cytokines are IL-1β, IL-6, TNFα, IL-2, IL-7, IL-15, and IL-21.

26. A method of promoting bone healing or increasing local bone formation, the method comprising administering to a subject in need thereof a therapeutically effective amount of the composition of any of claims 1 to 8 or 11 to 14 or the pharmaceutical composition of any of claims 9 to 10 or 15 to 16.

27. The method of any of claims 17 to 26, wherein the subject is a human.

28. The method of any of claims 17 to 26, wherein the subject has osteoarthritis.

29. The method of any of claims 17 to 26, wherein the subject has one or more injured joints.

30. The method of any of claims 17 to 26, wherein the subject has a skeletal defect.

31. The method of claim 30, wherein the skeletal defect is a large segmental bone defect.

32. The method of any of claims 17 to 26, wherein the subject has one or more bone fractures.

33. The method of any of claims 17 to 26, wherein the subject has one or more bone injuries.

34. The method of any of claims 17 to 26, wherein the composition or the pharmaceutical composition is formulated for local administration.

35. The method of any of claims 17 to 26, wherein the composition or the pharmaceutical composition is administered intraarticularly.
